(19) United States
(12) Patent Application Publication
(10) Pub. No.: US 2020/0155567 A1
(43) Pub. Date: May 21, 2020

(54) TREATMENT OF CANCERS USING A COMBINATION COMPRISING PARP INHIBITORS, TEMOZOLOMIDE AND/OR RADIATION THERAPY

(71) Applicant: BEIGENE, LTD., Camana Bay, Grand Cayman (KY)

(72) Inventors: Lai WANG, Beijing (CN); Zhiyu TANG, Beijing (CN); Lusong LUO, Beijing (CN); Min WEI, Beijing (CN); Amy PETERSON, Beijing (CN); Hexiang WANG, Beijing (CN); Bo REN, Beijing (CN); Changyou ZHOU, Princeton, NJ (US)

(21) Appl. No.: 16/630,103

(22) PCT Filed: Jul. 17, 2018
(86) PCT No.: PCT/CN2018/095911

§ 371 Date: Jan. 10, 2020

(30) Foreign Application Priority Data
Jul. 17, 2017 (CN) PCT/CN2017/093192

(51) Int. Cl. A61K 31/551; A61K 31/495; A61N 5/10; A61P 35/00; A61P 31/00

(52) U.S. Cl. CPC A61K 31/551; A61K 31/495; A61N 5/10; A61K 9/0053; A61P 31/00; A61N 2005/1098; A61P 35/00

(57) ABSTRACT

Disclosed herein is a method for the prevention, delay of progression or treatment of cancer in a subject, comprising administering to the subject in need thereof a PARP inhibitor, particularly, (R)-2-fluoro-10a-methyl-7, 8, 9, 10, 10a, 11-hexahydro-5, 6, 7a, 11-tetraazacyclohepta [def] cyclopenta [a] fluoren-4 (5H)-one, a sesqui-hydrate thereof, or a pharmaceutically acceptable salt thereof, in combination with temozolomide and/or radiation therapy. Also, disclosed a pharmaceutical combination comprising a PARP inhibitor, particularly, (R)-2-fluoro-10a-methyl-7, 8, 9, 10, 10a, 11-hexahydro-5, 6, 7a, 11-tetraazacyclohepta [def] cyclopenta [a] fluoren-4 (5H)-one, a sesqui-hydrate thereof, or a pharmaceutically acceptable salt thereof, in combination with temozolomide and the use thereof.

### CROSS-REFERENCE TO RELATED APPLICATIONS

This application claims the benefit of International Patent Application No. PCT/CN2017/093192 filed on Jul. 17, 2017, the disclosures of which is hereby incorporated by reference in its entirety for all purposes.

### FIELD OF THE INVENTION

Disclosed herein is a method for the prevention, delay of progression or treatment of cancer in a subject, comprising administering to the subject in need thereof a therapeutically effective amount of a PARP inhibitor, in combination with a therapeutically effective amount of temozolomide and/or radiation therapy.

### BACKGROUND OF THE INVENTION

One of the hallmarks and driving forces of cancer is genetic instability [Hanahan D and Weinberg R A, Hallmarks of cancer: the next generation. Cell, 2011. 144(5): p. 646-74]. Specifically in familial cancers, mutations in the breast cancer susceptibility BRCA1 and BRCA2 tumor suppressor genes, key players in homologous recombination (HR), have been associated with an increased risk of developing breast or ovarian cancer [Li X and Heyer W D, Homologous recombination in DNA repair and DNA damage tolerance. Cell Res, 2008. 18(1): p. 99-113]. It is in this patient population that inhibitors of poly (ADP-ribose) polymerase (PARP) have gained recent attention. PARP family members PARP1 and PARP2 play important roles in DNA replication, transcriptional regulation, and DNA damage repair [Rouleau M, Patel A, Hendzel M J, et al., PARP inhibition: PARP1 and beyond. Nat Rev Cancer, 2010. 10(4): p. 293-301]. In 2005, two breakthrough Nature papers showed that PARP inhibitors given alone could kill cancer cells with pre-existing DNA repair defects, specifically mutations in BRCA1/2 genes [Bryant HE, Schultz N, Thomas H D, et al., Specific killing of BRCA2-deficient tumours with inhibitors of poly(ADP-ribose) polymerase. Nature, 2005. 434(7035): p. 913-7; Farmer H, McCabe N, Lord C J, et al., Targeting the DNA repair defect in BRCA mutant cells as a therapeutic strategy. Nature, 2005. 434(7035): p. 917-21].

PARP inhibition and mutant BRCA were synthetically lethal in preclinical models, suggesting an elegant, targeted and minimally toxic way to treat patients.

Glioblastomas (GB or GBM), the most aggressive subtype of gliomas, harbor a range of oncogenic mutations. These mutations are associated with resistance to both chemotherapy and radiation therapy (RT). A substantial number of these genetic alterations affect key players in deoxyribonucleic acid (DNA) repair pathways.

The high frequency of genetic alterations in GB affecting DNA repair pathways suggests that DNA-damaging agents or agents interfering with DNA repair may be able to provide clinical benefit for GB patients. This hypothesis is supported by the current standard of care for GB patients but has not been adequately explored for other classes of drugs, such as inhibitors of poly(ADP-ribose) polymerase (PARP).

GB is the most common primary malignant brain tumor in adults with approximately 10,000 cases diagnosed annually in the United States (US) with a dismal prognosis despite aggressive treatment [CBTRUS statistical report: primary brain and central nervous system tumors diagnosed in the United States in 2004-2008. http://www.cbtrusorg. 2012 [updated 2012; cited 14 Aug. 2014]. Because of the infiltrative nature of GB, surgery alone is never curative. Therefore, the majority of patients are subsequently treated with RT, with or without chemotherapy. In 2005, Stupp and colleagues published a landmark study demonstrating a 2.5-month overall survival (OS) benefit with the addition of the alkylating agent temozolomide (TMZ) to surgery and RT [Stupp R, Mason W P, van den Bent M J, Weller M, Fisher B, Taphoorn M J, et al. Radiotherapy plus concomitant and adjuvant temozolomide for glioblastoma. N Engl J Med. 2005; 352(10):987-96]. The results of this large trial established the role of TMZ, along with maximal safe resection and RT, for the treatment of newly diagnosed GB patients <65 years old. Preliminary evidence that inactivation of the MGMT protein conferred sensitivity to TMZ [Esteller M, Garcia-Foncillas J, Andion E, Goodman S N, Hidalgo O F, Vanaclocha V, et al. Inactivation of the DNA-repair gene MGMT and the clinical response of gliomas to alkylating agents. N Engl J Med. 2000; 343(19):1350-4; Hegi M E, Diserens A C, Godard S, Dietrich P Y, Regli L, Ostermann S, et al. Clinical trial substantiates the predictive value of O-6-methylguanine-DNA methyltransferase promoter methylation in glioblastoma patients treated with temozolomide. Clin Cancer Res. 2004; 10(6): 1871-4] and TMZ's efficacy in recurrent glioma [Yung W K, Albright R E, Olson J, Fredericks R, Fink K, Prados M D, et al. A phase II study of temozolomide vs. procarbazine in patients with glioblastoma multiforme at first relapse. Br J Cancer. 2000; 83(5):588-93] served as supporting data for this large, randomized, Phase 3 trial. Subset analyses confirmed improved survival and sensitivity to TMZ for tumors deficient in MGMT (defined by MGMT promoter methylation) compared to those with adequate MGMT expression (defined by an unmethylated MGMT promoter) [Hegi M E, Diserens A C, Gorlia T, Hamou M F, de Tribolet N, Weller M, et al. MGMT gene silencing and benefit from temozolomide in glioblastoma. N Engl J Med. 2005; 352(10):997-1003]. However, significant regional debate remains with respect to whether incorporation of TMZ should be based upon MGMT methylation status alone [Newlands E S, Stevens M F, Wedge S R, Wheelhouse R T, Brock C. Temozolomide: a review of its discovery, chemical properties, pre-clinical development and clinical trials. Cancer Treat Rev. 1997; 23(1):35-61; Stupp R, Gander M, Leyvraz S, Newlands E. Current and future developments in the use of temozolomide for the treatment of brain tumours. Lancet Oncol. 2001; 2(9):552-60].

Very few cytotoxic drugs have demonstrated efficacy in GB, and this is thought to be due to, at least in part, the blood brain barrier preventing adequate delivery to these tumors.

TMZ and other alkylating agents, including the nitrosoureas carmustine and lomustine, are commonly used cytotoxic chemotherapies for newly diagnosed and recurrent GB. They induce apoptosis and cell death by methylating guanine at the O⁶ position, initiating a DSB in the DNA and cell cycle arrest. The MGMT protein removes the damaging alkyl groups from the O⁶ position of guanine and repairs the DNA. The alkylated protein is then degraded, requiring constant replenishment for DNA repair to be effective.

High expression of MGMT in cancer cells, including glioma cells, account for the predominant mechanism of resistance to alkylating agents. Consistent with this finding, lack of MGMT protein correlates with increased sensitivity to DNA-damaging agents, like temozolomide or XRT. The absence of MGMT protein in GB has been determined to be almost exclusively caused by methylation of the promoter of the MGMT gene [Hegi M E, Diserens A C, Gorlia T, Hamou M F, de Tribolet N, Weller M, et al. MGMT gene silencing and benefit from temozolomide in glioblastoma. N Engl J Med. 2005; 352(10):997-1003] referred to hereafter as ‘MGMT methylation’ or ‘methylated GB.’

Conversely, patients with an unmethylated MGMT promoter or higher levels of MGMT protein in their tumors are less likely to respond to alkylating agents resulting in shorter survival compared with patients with a methylated MGMT promoter or lower levels of MGMT protein (Table 1).

It is known that MGMT is inactivated after each reaction (i.e., suicide enzyme). Therefore, if the rate of DNA alkylation were to outpace the rate of MGMT protein synthesis, the enzyme could, in theory, be depleted. Several studies have shown that prolonged exposure to TMZ can deplete MGMT activity in blood cells, a process that could potentially increase the antitumor activity of the drug [Brandes A A, Tosoni A, Cavallo G, Bertorelle R, Gioia V, Franceschi E, Biscuola M, Blatt V, Crinò L, Ermani M, GICNO: Temozolomide 3 weeks on and 1 week off as first-line therapy for recurrent glioblastoma: phase II study from gruppo italiano cooperativo di neuro-oncologia (GICNO). Br J Cancer 95: 1155-1160, 2006; Gilbert M R, Wang M, Aldape K D, Stupp R, Hegi M E, Jaeckle K A, et al. Dose-dense temozolomide for newly diagnosed glioblastoma: a randomized phase III clinical trial. J Clin Oncol. 2013 Nov. 10; 31(32):4085-91; Strik H M, Buhk J H, Wrede A, Hoffmann A L, Bock H C, Christmann M, Kaina B.: Rechallenge with temozolomide with different scheduling is effective in recurrent malignant gliomas. Mol Med Report 1: 863-867, 2008; Tokher A W, Gerson S L, Denis L, Geyer C, Hammond L A, Patnaik A, Goetz A D, Schwartz G, Edwards T, Reyderman L, Statkevich P, Cutler D L, Rowinsky E K.: Marked inactivation of O6-alkylguanine-DNA alkyltransferase activity with protracted temozolomide schedules. Br J Cancer 88: 1004-1011, 20031 This resistance to TMZ remains a critical barrier to the effective treatment of glioblastoma.

Despite differences in the diagnostic methods used to detect MGMT methylation, studies consistently demonstrated improved outcomes for patients with methylated GB regardless of treatment, showed added benefit with TMZ and suggested that MGMT methylation is both prognostic and predictive (Table 1).

TABLE 1
O⁶-methylguanine-DNA Methyltransferase (MGMT) Status and Clinical
Benefit in Newly Diagnosed Glioblastoma

| | Age, | | PFS | | OS | |
|---|---|---|---|---|---|---|
| Study | median | Treatment | uMGMT | mMGMT | uMGMT | mMGMT |
| (Phase) | (range) | arm | (months) | (months) | (months) | (months) |
| Stupp | 56 | Radiation | 4.4 | 5.9 | 11.8 | 15.3 |
| (Phase 3) | (19-71) | Chemotherapy | 5.3 | 10.3 | 12.7 | 21.7 |
| NOA-08 | 72 | Radiation | 4.6 (EFS) | 4.6 (EFS) | 10.4 | 9.6 |
| (Phase 3) | (66-84) | Chemotherapy | 3.3 (EFS) | 8.4 (EFS) | 7 | Not |
| | | | | | | reached |
| Nordic | 70 | Radiation | Not | Not | 7.0 | 8.2 |
| (Phase 3) | (60-88) | | collected | collected | | |
| | | Chemotherapy | Not | Not | 6.8 | 9.7 |
| | | | collected | collected | | |
| ANOCEF | 77 | Chemotherapy | 2.6 | 6.0 | 4.4 | 7.2 |
| (Phase 2) | (80-87) | | | | | |
| Brandes | 68 | Chemoradiation | 9.5 | 22.9 | 13.7 | Not |
| (Phase 2) | (65-82) | | | | | reached |
| German | 74 | Radiation | 5.2 | 4.5 | 8.8 | 7.8 |
| Glioma | (70-86.6) | Chemotherapy | 0.5 | 6.8 | 2.6 | 7.2 |
| Network | | Chemoradiation | 7.2 | 7.3 | 10.4 | 13.1 |
| (Observational) | | | | | | |

Sources: General: Chen et al. Eur J Neurol 2013 [Chen C, Xu T, Lu Y, Chen J, Wu S. The efficacy of temozolomide for recurrent glioblastoma multiforme. Eur J Neurol. 2013 February; 20(2):223-30];
Stupp: Stupp et al. N Engl J Med. 2005 [Stupp R, Mason W P, van den Bent M J, Weller M, Fisher B, Taphoorn M J, et al. Radiotherapy plus concomitant and adjuvant temozolomide for glioblastoma. N Engl J Med. 2005; 352(10):987-96];
NOA-08: Wick et al. Lancet Oncol 2012 [Wick W, Platten M, Meisner C, Felsberg J, Tabatabai G, Simon M, et al. Temozolomide chemotherapy alone versus radiotherapy alone for malignant astrocytoma in the elderly: the NOA-08 randomised, phase 3 trial. Lancet Oncol. 2012 July; 13(7):707-15];
Nordic: Malmström et al. Lancet Oncol 2012 [Malmström A, Gronberg B H, Marosi C, Stupp R, Frappaz D, Schultz H, et al. Temozolomide versus standard 6-week radiotherapy versus hypofractionated radiotherapy in patients older than 60 years with glioblastoma: the Nordic randomised, phase 3 trial. Lancet Oncol. 2012 September; 13(9):916-26];
ANOCEF: Gállego Pérez-Larraya et al. J Clin Oncol 2011 [Gállego Pérez-Larraya J, Ducray F, Chinot O, Catry-Thomas I, Taillandier L, Guillamo J S, et al. Temozolomide in elderly patients with newly diagnosed glioblastoma and poor performance status: an ANOCEF phase II trial. J Clin Oncol. 2011 Aug. 1; 29(22):3050-5];
Brandes: Brandes et al. Cancer. 2009 [Brandes A A, Franceschi E, Tosoni A, Benevento F, Scopece L, Mazzocchi V, et al. Temozolomide concomitant and adjuvant to radiotherapy in elderly patients with glioblastoma: correlation with MGMT promoter methylation status. Cancer. 2009 Aug. 1; 115(15):3512-8];
GGN: Weller et al. J Clin Oncol 2009 [Weller M, Felsberg J, Hartmann C, Berger H, Steinbach J P, Schramm J, et al. Molecular predictors of progression-free and overall survival in patients with newly diagnosed glioblastoma: a prospective translational study of the German Glioma Network. J Clin Oncol. 2009 Dec. 1; 27(34):5743-50].

EFS=event-free survival; mMGMT=methylated O⁶-methylguanine-DNA methyltransferase promoter; OS=overall survival; PFS=progression-free survival; uMGMT=unmethylated O⁶-methylguanine-DNA methyltransferase promoter

In efforts to examine newly diagnosed glioblastoma patients with an unmethylated MGMT promoter or higher levels of MGMT protein in their tumors who are assumed to be less likely to respond to alkylating agents, trials have been conducted to support the omission of TMZ therapy. Multiple European-led trials have used RT alone, over RT with chemotherapy, as the comparative arm in trials of novel targets to replace TMZ [Herrlinger U, Schaefer N, Steinbach J P, Weyerbrock A, Hau P, Goldbrunner R, et al. Survival and quality of life in the randomized, multicenter GLARIUS trial investigating bevacizumab/irinotecan versus standard temozolomide in newly diagnosed, MGMT-non-methylated glioblastoma patients. J Clin Oncol. 2014; 32 Suppl 5:2042; Wick W, Gorlia T, van den Bent M J, Vecht C J, Steuve J, Brandes A A, et al. Radiation therapy and concurrent plus adjuvant temsirolimus (CCI-779) versus chemo-irradiation with temozolomide in newly diagnosed glioblastoma without methylation of the MGMT gene promoter. J Clin Oncol. 2014; 32 Suppl 5:2003; Wick W, Steinbach J P, Platten M, Hartmann C, Wenz F, von Deimling A, et al. Enzastaurin before and concomitant with radiation therapy, followed by enzastaurin maintenance therapy, in patients with newly diagnosed glioblastoma without MGMT promoter hypermethylation. Neuro Oncol. 2013; 15(10):1405-12]. The GLARIUS trial, a randomized Phase 2 study of irinotecan, bevacizumab and RT versus TMZ and RT in newly diagnosed unmethylated GB found a significantly prolonged median progression-free survival (mPFS) of 9.7 months in the experimental arm versus 5.9 months in the standard arm [Herrlinger U, Schaefer N, Steinbach J P, Weyerbrock A, Hau P, Goldbrunner R, et al. Survival and quality of life in the randomized, multicenter GLARIUS trial investigating bevacizumab/irinotecan versus standard temozolomide in newly diagnosed, MGMT-non-methylated glioblastoma patients. J Clin Oncol. 2014; 32 Suppl 5:2042]. Despite the mPFS benefit observed in this study, the median overall survival (mOS) between the two groups was comparable (16.6 months in the experimental arm compared with 17.3 months in the TMZ arm). This suggested that it is reasonable to omit TMZ in newly diagnosed unmethylated patients without adversely impacting the patients' survival [Herrlinger U, Schaefer N, Steinbach J P, Weyerbrock A, Hau P, Goldbrunner R, et al. Survival and quality of life in the randomized, multicenter GLARIUS trial investigating bevacizumab/irinotecan versus standard temozolomide in newly diagnosed, MGMT-non-methylated glioblastoma patients. J Clin Oncol. 2014; 32 Suppl 5:2042].

In the recurrent setting, MGMT methylation status has yet to guide treatment. Preliminary data suggested that prolonged exposure to TMZ may suppress MGMT activity, therefore making the cells more susceptible than the standard 5-day regimen (Days 1 to 5 of a 28-day cycle) [Hegi M E, Diserens A C, Gorlia T, Hamou M F, de Tribolet N, Weller M, et al. MGMT gene silencing and benefit from temozolomide in glioblastoma. N Engl J Med. 2005; 352(10):997-1003]. This hypothesis led to a series of studies of dose-dense schedules to prevent repletion of MGMT and improve sensitivity to TMZ and shift the outcome of unmethylated GB patients toward methylated GB patients [Perry J R, Bélanger K, Mason W P, Fulton D, Kavan P, Easaw J, et al. Phase II trial of continuous dose-intense temozolomide in recurrent malignant glioma: RESCUE study. J Clin Oncol. 2010 Apr. 20; 28(12):2051-7; Weller M, Cloughesy T, Perry J R, Wick W. Standards of care for treatment of recurrent glioblastoma—are we there yet? Neuro Oncol. 2013 January; 15(1):4-27; Weller M, Tabatabai G, Reifenberger G, et al. Dose-intensified rechallenge with temozolomide: one week on/one week off versus 3 weeks on/one week off in patients with progressive or recurrent glioblastoma. J Clin Oncol. 2010; 28(Suppl. 15) Abstract TPS154]. A large, randomized trial of dose-dense TMZ (21 days on and 7 days off at 100 mg/m²) in the adjuvant setting (RTOG 0525) in newly diagnosed GB patients resolved the conflicting data on timing of standard temozolomide and efficacy of alternative dosing. No improvement was seen in mPFS or mOS with dose-dense TMZ, regardless of MGMT promoter methylation status [Gilbert M R, Wang M, Aldape K D, Stupp R, Hegi M E, Jaeckle K A, et al. Dose-dense temozolomide for newly diagnosed glioblastoma: a randomized phase III clinical trial. J Clin Oncol. 2013 Nov. 10; 31(32):4085-91]. These findings, in addition to results from dose-dense TMZ at recurrence [Wick W, Platten M, Weller M. New (alternative) temozolomide regimens for the treatment of glioma. Neuro Oncol. 2009 February; 11(1):69-79], suggest that unmethylated tumors cannot be ‘sensitized’ to TMZ by solely intensifying the dose.

As summarized in Table 1, GBs have a high prevalence of genetic alterations affecting DNA repair pathways, raising the possibility that PARP inhibitors may be able to contribute to clinical benefit for GB patients. Whereas this hypothesis has not been adequately explored in the clinic, nonclinical data for one such alteration, PTEN loss, support the concept of synthetic lethality of PARP inhibition with other pathways aside from BRCA1/2. Deletions in chromosome 10 encompassing the PTEN gene have been frequently observed in GB [Endersby R, Baker S J. PTEN signaling in brain: neuropathology and tumorigenesis. Oncogene. 2008 Sep. 18; 27(41):5416-30; Li L, Ross A H. Why is PTEN an important tumor suppressor? J Cell Biochem. 2007 Dec. 15; 102(6):1368-74, 34] and have been estimated to occur in about one third of GBs [Smith J S, Tachibana I, Passe S M, Huntley B K, Borell T J, Iturria N, O'Fallon J R, et al. PTEN Mutation, EGFR Amplification, and Outcome in Patients With Anaplastic Astrocytoma and Glioblastoma Multiforme. J Nati Cancer Inst. 2001; 93 (16): 1246-1256]. PTEN is a lipid phosphatase with a role in dampening PI3K/Akt signaling, and PTEN loss results in PI3K/Akt pathway hyperactivation. However, PTEN also plays a role in the maintenance of genome stability as demonstrated using mouse embryonic PTEN+/− cells. This phenotype was related to a defect in the regulation of the expression of RAD51, an important HR component [Shen W H, Balajee A S, Wang J, Wu H, Eng C, Pandolfi P P, et al. Essential role for nuclear PTEN in maintaining chromosomal integrity. Cell. 2007 Jan. 12; 128(1):157-70]. Synthetic lethality of PTEN loss and PARP inhibition is supported by data that the PARP inhibitor veliparib is very effective in reducing the survival of PTEN+/− human GB cell lines, whereas astrocytes with the intact PTEN gene are less sensitive to veliparib. PTEN-deficient astrocytes and GB cells were also more sensitive to the methylating agent N-methyl-N′-nitro-N-nitrosoguanidine (MNNG) with a mechanism of action very similar to TMZ [McEllin B, Camacho C V, Mukherjee B, Hahm B, Tomimatsu N, Bachoo R M, et al. PTEN loss compromises homologous recombination repair in astrocytes: implications for glioblastoma therapy with temozolomide or poly(ADP-ribose) polymerase inhibitors. Cancer Res. 2010 Jul. 1; 70(13):5457-64]. These data suggest that GBs with defects in DNA repair pathways may be sensitive to PARP inhibition, in particular when combined with DNA damaging agents.

PARP-1 and PARP-2 have a key role in the base excision repair (BER) of N-methylpurines (N7-methylguanine and N3-methyladenine) that are generated by TMZ. In the presence of a functional BER system these damaged bases are promptly repaired and limit TMZ cytotoxicity. The first step of the BER process is the excision of the modified base by N-methylpurine glycosylase (MPG) resulting in an apurinic/apyrimidinic (AP) site that is subsequently cleaved by apurinic/apyrimidinic endonuclease. The resultant DNA nicks are finally repaired by the coordinate intervention of PARP-1, DNA polymerase, XRCC1 and ligase III. Inhibition of PARP activity hampers PARylation of PARP-1 and PARP-2, interrupting the completion of the repair process mediated by BER [Kim Y J, Wilson D M 3rd. Overview of base excision repair biochemistry. Curr Mol Pharmacol. 2012 January; 5(1):3-13]. Combining PARP inhibition with DNA-damaging TMZ leads to increased DNA damage that results in apoptosis and/or growth arrest. Repeated treatments with TMZ and PARP inhibitors also downregulate transcription and delay recovery of BER components in tumor cells [Tentori, L.; Turriziani, M.; Franco, D.; Serafino, A.; Levati, L.; Roy, R.; Bonmassar, E.; Graziani, G. Treatment with temozolomide and poly(ADP-ribose) polymerase inhibitors induces early apoptosis and increases base excision repair gene transcripts in leukemic cells resistant to triazene compounds. Leukemia 1999, 13, 901-909; Tentori, L.; Portarena, I.; Vernole, P.; De Fabritiis, P.; Madaio, R.; Balduzzi, A.; Roy, R.; Bonmassar, E.; Graziani, G. Effects of single or split exposure of leukemic cells to temozolomide, combined with poly(ADP-ribose) polymerase inhibitors on cell growth, chromosomal aberrations and base excision repair components. Cancer Chemother. Pharmacol. 2001, 47, 361-369]. This mechanism might further enhance the cytotoxic effects of TMZ combined with a PARP inhibitor.

Since the discovery of synthetic lethality of PARP inhibitors in BRCA-deficient cells, accumulation of unrepaired SSBs resulting from catalytic PARP inhibition has been considered central to the mechanism of action of PARP inhibitors. More recently, it has been demonstrated that PARP inhibitors also trap PARP1- and PARP2-DNA complexes at DNA damage sites and that PARP trapping can be more cytotoxic than unrepaired SSBs [Kedar P S, Stefanick D F, Horton J K, Wilson S H. Increased PARP-1 association with DNA in alkylation damaged, PARP-inhibited mouse fibroblasts. Mol Cancer Res 2012; 10:360-8. Murai J, Huang S Y, Das B B, Renaud A, Zhang Y, Doroshow J H, et al. Trapping of PARP1 and PARP2 by Clinical PARP Inhibitors. Cancer Res. 2012; 72:5588-5599; Murai J, Huang S Y, Renaud A, Zhang Y, Ji J, Takeda S, et al. Stereospecific PARP Trapping by BMN 673 and Comparison with Olaparib and Rucaparib. Mol Cancer Ther 2014; 13:433-443; Fojo T and Bates S. Mechanisms of resistance to PARP inhibitors—three and counting. Cancer Discov 2013; 3:20-23]. Murai and colleagues investigated whether PARP trapping is important for chemotherapy combinations that are currently being studied in the clinic. To this purpose, a PARP inhibitor with potent PARP-trapping activity, olaparib, was compared to a PARP inhibitor with similar catalytic PARP inhibition but significantly less PARP-trapping activity, veliparib. Both drugs showed highly synergistic effects with the toposisomerase I inhibitor camptothecin, consistent with catalytic PARP inhibition being important for the activity of this combination. However, in combination with the alkylating agent TMZ, olaparib was significantly more effective than veliparib indicating that PARP trapping was essential for the combination activity [Murai J, Zhang Y, Morris J, Ji J, Takeda S, Doroshow J H, Pommier Y. Rationale for Poly(ADP-ribose) Polymerase (PARP) Inhibitors in Combination Therapy with Camptothecins or Temozolomide Based on PARP Trapping versus Catalytic Inhibitions. J Pharmacol Exp Ther 349:408-416, June 2014]. Gill and colleagues similarly showed that sensitivity to a PARP inhibitor was due to DNA trapping activity and that TMZ-mediated potentiation of PARP inhibitor activity was associated with enhanced trapping of PARP1-DNA complexes [Gill S J, Travers J, Pshenichnaya I, Kogera F A, Barthorpe S, Mironenko T. Combinations of PARP Inhibitors with Temozolomide Drive PARP1 Trapping and Apoptosis in Ewing's Sarcoma. PLoS One. 2015 Oct. 27; 10(10)]. These data suggest that combinations of TMZ with PARP inhibitors with potent DNA-trapping activity may be of particular interest.

In glioma cells, pharmacological modulation of PARP activity increased growth inhibition by TMZ in both p53-wild-type and p53-mutant glioblastoma cells and markedly lowered the TMZ IC50 to levels below the concentration of TMZ that can be detected in the plasma or brain of treated patients. The most pronounced effect was observed in tumor cells resistant to TMZ due to high MGMT levels or to MMR deficiency. In fact, in short-term primary cultures of glioma cells derived from surgical specimens, the enhancement of chemosensitivity to TMZ induced by a PARP inhibitor was especially evident in MGMT-proficient cells. Moreover, in an MMR-deficient glioma cell line, in which an MGMT inhibitor would have been ineffective, the combination of TMZ with the PARP inhibitor reverted resistance to the methylating compound [Tentori, L.; Portarena, I.; Vernole, P.; De Fabritiis, P.; Madaio, R.; Balduzzi, A.; Roy, R.; Bonmassar, E.; Graziani, G. Effects of single or split exposure of leukemic cells to temozolomide, combined with poly(ADP-ribose) polymerase inhibitors on cell growth, chromosomal aberrations and base excision repair components. Cancer Chemother. Pharmacol. 2001, 47, 361-369; Tentori, L.; Portarena, I.; Torino, F.; Scerrati, M.; Navarra, P.; Graziani, G. Poly(ADP-ribose) polymerase inhibitor increases growth inhibition and reduces G(2)/M cell accumulation induced by temozolomide in malignant glioma cells. Glia 2002, 40, 44-54; Tentori, L.; Leonetti, C.; Scarsella, M; D'Amati, G.; Vergati, M; Portarena, I.; Xu, W; Kalish, V.; Zupi, G.; Zhang, J.; Graziani, G. Systemic administration of GPI 15427, a novel poly(ADP-ribose) polymerase-1 inhibitor, increases the antitumour activity of temozolomide against intracranial melanoma, glioma, lymphoma. Clin. Cancer Res. 2003, 9, 5370-5379.]. These data suggest that GB patients who derive less benefit from current standard of care because of lack of MGMT promoter methylation may benefit from a combination regimen that includes a PARP inhibitor.

Several clinical studies have been conducted with PARP inhibitors (olaparib, rucaparib, and veliparib) in combination with TMZ. To determine the MTD in these studies, TMZ was administered at standard doses (150 to 1000 mg/m²) with increasing doses of the PARP inhibitor. All studies experienced the challenge of significant myelosuppression as dose-limiting toxicities, and observed anti-tumor activity was only modest [Gabrielson A, Tesfaye A A, Marshall J L, Pishvaian M J, Smaglo B, Jha R, Dorsch-Vogel K, Wang H, He A R. Phase II study of temozolomide and veliparib combination therapy for sorafenib-refractory advanced hepatocellular carcinoma. Cancer Chemother Pharmacol. 2015; 76(5):1073-9; Gojo I, Beumer J H, Pratz K W, McDevitt M A, Baer M R, Blackford A L, et al. A Phase 1 Study of the PARP Inhibitor Veliparib in Combination with Temozolomide in Acute Myeloid Leukemia. Clin Cancer Res. 2017; 23(3):697-706; Hussain M, Carducci M A, Slovin S, Cetnar J, Qian J, McKeegan E M, et al. Targeting DNA repair with combination veliparib (ABT-888) and temozolomide in patients with metastatic castration-resistant prostate cancer. Invest New Drugs. 2014; 32(5):904-12; Middleton M R, Friedlander P, Hamid O, Daud A, Plummer R, Falotico N, et al. Randomized phase II study evaluating veliparib (ABT-888) with temozolomide in patients with metastatic melanoma. Ann Oncol. 2015; 26(10):2173-9; Plummer R, Lorigan P, Steven N, Scott L, Middleton M R, Wilson R H, et al. A phase II study of the potent PARP inhibitor, Rucaparib (PF-01367338, AGO14699), with temozolomide in patients with metastatic melanoma demonstrating evidence of chemopotentiation. Cancer Chemother Pharmacol. 2013; 71(5): 1191-9; Su J M, Thompson P, Adesina A, Li XN, Kilburn L, Onar-Thomas A, et al. A phase I trial of veliparib (ABT-888) and temozolomide in children with recurrent CNS tumors: a pediatric brain tumor consortium report. Neuro Oncol. 2014; 16(12):1661-8]. This is contrasted by a recent study for talazoparib, a PARP inhibitor with very good DNA-trapping activity. Standard doses of talazoparib (0.5-1 mg) were administered with low doses of TMZ in subjects with non-BRCA1/2-mutated cancers [Wainberg Z A, Hecht J R, Konecny G E, Goldman J W, Sadeghi S, Chmielowski B, et al. Safety and efficacy results from a phase I dose-escalation trial of the PARP inhibitor talazoparib in combination with either temozolomide or irinotecan in patients with advanced malignancies. Abstract CT011; AACR Annual Meeting 2016]. The starting dose of TMZ was 25 mg/m², approximately 12.5% of the therapeutic dose, and the MTD was determined as 1 mg talazoparib plus 37 mg/m² of TMZ. This regimen was better tolerated than reported for prior studies, with less thrombocytopenia and neutropenia. Furthermore, promising efficacy was observed with 11 subjects (61%) experiencing either a partial response or stable disease. These preliminary clinical results are in support of the hypothesis that PARP inhibitors with strong DNA-trapping activity may only require relatively low TMZ dose levels to exert their anti-tumor activity.

Ionizing radiation used in the clinical treatment of GB generates mostly single-strand breaks (SSBs) and to a minor extent DSBs. Single-strand breaks are repaired through the BER pathway, operating via either the short patch or the long patch repair sub-pathways, which differ in the size of the repair patch and the enzymes involved. A PARP-1 role in the short patch is well established, but its contribution in the long patch is still unclear. In non-replicating cells, PARP inhibition only delays the repair of SSBs induced by radiation with a minimal impact on cell survival. On the contrary, PARP inhibition markedly enhances radiosensitivity of proliferating cells since unrepaired SSBs collide with the DNA replication machinery, generating DSBs. Thus, PARP inhibitors have the potential to increase the anti-tumor effect of RT by preventing DNA damage repair and increasing cytotoxic DNA damage [Godon, C.; Cordelieres, F. P.; Biard, D.; Giocanti, N.; Mégnin-Chanet, F.; Hall, J.; Favaudon, V. PARP inhibition versus PARP-1 silencing: different outcomes in terms of single-strand break repair and radiation susceptibility. Nucleic Acids Res. 2008, 36, 4454-4464; Noel, G.; Godon, C.; Fernet, M; Giocanti, N.; Mégnin-Chanet, F.; Favaudon, V. Radiosensitization by the poly(ADPribose) polymerase inhibitor 4-amino-1,8-naphthalimide is specific of the S phase of the cell cycle and involves arrest of DNA synthesis. Mol. Cancer Ther. 2006, 5, 564-574; Dungey, F. A.; Loser, D. A.; Chalmers, A. J. Replicationdependent radiosensitization of human glioma cells by inhibition of poly(ADP-Ribose) polymerase: replication-dependent radiosensitization of human glioma cells by inhibition of poly(ADP-ribose) polymerase: Mechanisms and therapeutic Int. J. Radiat. Biol. Phys. 2008, 72, 1188-1197].

Despite various clinical trials of combining Parp inhibitors such as Veliparib, Olaparib, etc, with TMZ and/or radiation have been attempted for the treatment of solid tumors, no significant clinical benefits have been disclosed, especially in the treatment of brain cancers such as GBM. The failures of those clinical trials may be probably attributed to the unexpected myelosuppression adverse effect intensified by combination therapy so the patients can't take the full benefit of combination treatment of Parp inhibitors, TMZ and/or radiation. It is highly desirable to discover an efficacious combination treatment without severe adverse effects such as myelosuppression.

WO2013/097225A1 disclosed a series of PARP inhibitor having the following general Formula (I) or a stereoisomer thereof, or a pharmaceutically acceptable salt thereof,

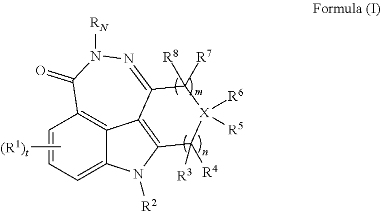

In particularly, (R)-2-fluoro-10a-methyl-7,8,9,10,10a,11-hexahydro-5,6,7a,11-tetraazacyclohepta[def]cyclopenta[a]fluoren-4(5H)-one (hereinafter Compound A), disclosed in WO2013/097225A1, has highly selective and potent PARP1/2 inhibitory activities.

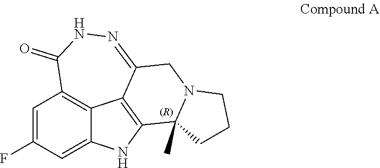

WO2017032289A also discloses crystalline forms of Compound A, particularly, (R)-2-fluoro-10a-methyl-7,8,9,10,10a,11-hexahydro-5,6,7a,11-tetraazacyclohepta[def]cyclopenta[a]fluoren-4(5H)-one sesqui-hydrate (hereinafter Compound B).

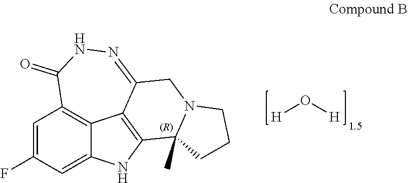

The inventors of the present application have unexpectedly found in the preclinical and clinical studies that the combination therapy of a particular PARP inhibitor (in particular, the above-mentioned Compound A or Compound B) with temozolomide and/or radiation demonstrates better anti-tumor activity than the monotherapy of each of the above active pharmaceutical agent alone in the treatment of solid cancers, particularly in the treatment of GBM. More specifically, the inventors of the present application unexpectedly found that the combination therapy disclosed herein does not produce severe myelosuppression toxicity as reported in the other combinations; and the claimed combination therapy provides a longer survival time and/or a constant reduced tumor volume for the patients with GBM.

### SUMMARY OF THE INVENTION

Disclosed herein is a method for the prevention, delay of progression or treatment of solid cancer, particularly brain cancers, in a subject, comprising administering to the subject in need thereof a PARP inhibitor (in particularly (R)-2-fluoro-10 a-methyl-7 ,8,9,10,10 a,11-hexahydro -5,6,7a,11-tetraazacyclohepta[def]cyclopenta[a]fluoren-4(5H)-one or a pharmaceutically acceptable salt thereof, (R)-2-fluoro-10a-methyl-7,8,9,10,10a,11-hexahydro-5,6,7a,11-tetraazacyclohepta[def]cyclopenta[a]fluoren-4(5H)-one sesqui-hydrate) in combination with temozolomide and/or radiation therapy. Disclosed herein is also a pharmaceutical combination comprising a PARP inhibitor (in particularly (R)-2-fluoro-10a-methyl-7,8,9,10,10a,11-hexahydro -5,6,7a,11-tetraazacyclohepta[def]cyclopenta[a]fluoren-4(5H)-one or a pharmaceutically acceptable salt thereof, (R)-2-fluoro-10a-methyl-7,8,9,10,10a,11-hexahydro-5,6,7a,11-tetraazacyclohepta[def]cyclopenta[a]fluoren-4(5H)-one sesqui-hydrate) in combination with temozolomide and/or radiation therapy and the use thereof.

In a first aspect, disclosed herein is a method for the prevention, delay of progression or treatment of cancer in a subject, comprising administering to the subject in need thereof a therapeutically effective amount of a PARP inhibitor of Formula (I) or a stereoisomer thereof, a pharmaceutically acceptable salt thereof or a solvate thereof, in combination with a therapeutically effective amount of temozolomide and/or radiation therapy.

In a second aspect, disclosed herein is a pharmaceutical combination for use in the prevention, delay of progression or treatment of cancer, comprising a PARP inhibitor of Formula

(I) or a stereoisomer thereof, a pharmaceutically acceptable salt thereof or a solvate thereof, in combination with a therapeutically effective amount of temozolomide and/or radiation therapy.

In a third aspect, disclosed herein is a PARP inhibitor of Formula (I) or a stereoisomer thereof, a pharmaceutically acceptable salt thereof or a solvate thereof, for use in the prevention, delay of progression or treatment of cancer in combination with a therapeutically effective amount of temozolomide and/or radiation therapy. In one embodiment of this aspect, disclosed herein is temozolomide and/or radiation therapy for use in the prevention, delay of progression or treatment of cancer in combination with a PARP inhibitor of Formula (I) or a stereoisomer thereof, a pharmaceutically acceptable salts thereof or a solvate thereof.

In a fourth aspect, disclosed herein is a use of a pharmaceutical combination in the manufacture of a medicament for use in the prevention, delay of progression or treatment of cancer, said pharmaceutical combination comprising a PARP inhibitor of Formula (I) or a stereoisomer thereof, a pharmaceutically acceptable salt thereof, or a solvate thereof, and temozolomide.

In a fifth aspect, disclosed herein is an article of manufacture, or “kit” comprising a first container, a second container and a package insert, wherein the first container comprises at least one dose of a medicament comprising a PARP inhibitor of Formula (I) or a stereoisomer thereof, a pharmaceutically acceptable salt thereof, or a solvate thereof; the second container comprises at least one dose of a medicament comprising temozolomide, and the package insert comprises instructions for treating cancer a subject using the medicaments.

In some embodiments, the PARP inhibitor is Compound A. In other embodiments, the PARP inhibitor is Compound B.

In some embodiments, the PARP inhibitor is administered continuously or intermittently during the treatment cycle.

In some embodiments, the method comprises 1 to 3 treatment cycles, and each treatment cycle comprises 1 to 4 weeks.

In some embodiments, temozolomid is administrated at standard dosing schedule, including 20 mg-120 mg once a day (QD).

In some embodiments, the radiation therapy administered QD×5 days/week for 6 to 7 weeks with 1.8 to 2 Gy/fraction for a total dose of up to 60 Gy.

In some embodiments, the amount of the PARP inhibitor in the maintenance phase is 1-120 mg, preferably, 5-120 mg (in terms of the parent compound) with the administration frequency of once to twice a day; preferably, the administered dosage of the PARP inhibitor is 5-80 mg (in terms of the parent compound), and the administration frequency is twice a day (BID). In other embodiments, the PARP inhibitor is administrated at a dose of 60 mg twice daily (BID).

The method and pharmaceutical combination disclosed herein, as a combination therapy, produce more efficacious anti-tumor response than either single agent alone.

In an embodiment of each of the above aspects, the cancer is solid cancers. In some embodiments, the cancer is selected from colorectal cancer, gastric cancer, small cell lung cancer (SCLC), breast cancer, ovarian cancer, fallopian tube carcinoma, peritoneal carcinoma, melanoma, glioblastoma or lymphoma. In some embodiments, the cancer is glioblastoma with unmethylated MGMT promoter. In some embodiments, the cancer is recurrent/refractory glioblastoma. In some embodiment, the cancer is locally advanced or metastatic solid tumors or newly diagnosed or recurrent/refractory glioblastoma.

In an embodiment of each of the above five aspects, the PARP inhibitor is (R)-2-fluoro -10a-methyl-7,8,9,10,10a,11 -hexahydro-5,6,7a,11-tetraazacyclohepta[def]cyclopenta[a]fluoren -4(5H)-one (Compound A), or a pharmaceutically acceptable salt thereof. In an embodiment of each of the above five aspects, the PARP inhibitor is (R)-2-fluoro-10a-methyl-7,8,9,10,10a,11-hexahydro-5,6,7a,11-tetraazacyclohepta[def]cyclopenta[a]fluoren-4(5H)-one sesqui-hydrate (Compound B). In an embodiment of each of the above five aspects, the PARP inhibitor and temozolomide and/or radiation therapy are administered simultaneously, sequentially or intermittently.

### BRIEF DESCRIPTION OF THE DRAWING

FIG. 1 shows the X-ray diffraction pattern of crystal Compound B.

FIG. 2 shows the ¹H-NMR of crystal Compound B.

FIG. 3 shows the ¹³C-NMR of crystal Compound B.

FIG. 4 shows the combination activity of Compound B and temozolomide in H209 Small Cell Lung Cancer Xenograft Model.

FIG. 5 shows the combination activity of Compound B and temozolomide in H209-T Intracranial Model.

### DETAILED DESCRIPTION OF THE INVENTION

### Definitions

Unless specifically defined elsewhere in this document, all other technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

Throughout this specification and the claims which follow, unless the context requires otherwise, the word “comprise”, and variations such as “comprises” and “comprising”, will be understood to imply the inclusion of an active agent (e.g., a mAb or a Btk inhibitor) or a stated amino acid sequence, but not the exclusion of any other active ingredient or amino acid sequence. When used herein the term “comprising” can be interchangeable with the term “containing” or “including”.

The term “alkyl” herein refers to a hydrocarbon group selected from linear and branched saturated hydrocarbon groups comprising from 1 to 18, such as from 1 to 12, further such as from 1 to 6, carbon atoms. Examples of the alkyl group can be selected from methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group can be selected from 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1 -butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂) and 3,3-dimethyl-2-butyl (—CH(CH₃)C(CH₃)₃ groups.

The term “alkenyl” herein refers to a hydrocarbon group selected from linear and branched hydrocarbon groups comprising at least one C═C double bond and from 2 to 18, such as from 2 to 6, carbon atoms. Examples of the alkenyl group may be selected from ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” herein refers to a hydrocarbon group selected from linear and branched hydrocarbon group, comprising at least one C≡C triple bond and from 2 to 18, such as from 2 to 6, carbon atoms. Examples of the alkynyl group include ethynyl (—C≡CH), 1-propynyl (—C≡CCH₃), 2-propynyl (propargyl, —CH₂C≡CH), 1-butynyl, 2-butynyl, and 3-butynyl groups.

The term “cycloalkyl” herein refers to a hydrocarbon group selected from saturated and partially unsaturated cyclic hydrocarbon groups, comprising monocyclic and polycyclic (e.g., bicyclic and tricyclic) groups. For example, the cycloalkyl group may comprise from 3 to 12, such as 3 to 8, further such as 3 to 6, 3 to 5, or 3 to 4 carbon atoms. Even further for example, the cycloalkyl group may be selected from monocyclic group comprising from 3 to 12, such as 3 to 8, 3 to 6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having from 7 to 12 ring atoms arranged as a bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from:

5- and 6-membered carbocyclic aromatic rings, for example, phenyl;
bicyclic ring systems such as 7 to 12 membered bicyclic ring systems wherein at least one ring is carbocyclic and aromatic, selected, for example, from naphthalene and indane; and
tricyclic ring systems such as 10 to 15 membered tricyclic ring systems wherein at least one ring is carbocyclic and aromatic, for example, fluorene.

For example, the aryl group is selected from 5 and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings are fused with a heterocyclic aromatic ring, the resulting ring system is heteroaryl, not aryl, as defined herein.

The term “arylalkyl” herein refers to an alkyl group as defined above substituted by an aryl group as defined above.

The term “halogen” or “halo” herein refers to F, Cl, Br or I.

The term “heteroaryl” herein refers to a group selected from:

5- to 7-membered aromatic, monocyclic rings comprising at least one heteroatom, for example, from 1 to 4, or, in some embodiments, from 1 to 3, heteroatoms, selected from N, O, and S, with the remaining ring atoms being carbon;
8- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” herein refers to a ring selected from 4- to 12-membered monocyclic, bicyclic and tricyclic, saturated and partially unsaturated rings comprising at least one carbon atoms in addition to at least one heteroatom, such as from 1-4 heteroatoms, further such as from 1-3, or further such as 1 or 2 heteroatoms, selected from oxygen, sulfur, and nitrogen. “Heterocycle” herein also refers to a 5- to 7-membered heterocyclic ring comprising at least one heteroatom selected from N, O, and S fused with 5-, 6-, and /or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl. “Heterocycle” herein also refers to an aliphatic spirocyclic ring comprising at least one heteroatom selected from N, O, and S, provided that the point of attachment is at the heterocyclic ring. The rings may be saturated or have at least one double bond (i.e., partially unsaturated). The heterocycle may be substituted with oxo. The point of the attachment may be carbon or heteroatom in the heterocyclic ring. A heterocycle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl,1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinyl, imidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. A substituted heterocycle also includes a ring system substituted with one or more oxo moieties, such as piperidinyl N-oxide, morpholinyl-N-oxide, 1-oxo-l-thiomorpholinyl and 1,1-dioxo-1-thiomorpholinyl.

Compounds described herein may contain an asymmetric center and may thus exist as enantiomers. Where the compounds described herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

The term “substantially pure” as used herein means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoiosomer(s).

When compounds described herein contain olefinic double bonds, unless specified otherwise, such double bonds are meant to include both E and Z geometric isomers.

Some of the compounds described herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)ₙ—COOH, wherein n is selected from 0 to 4. Similarly, examples of pharmaceutically acceptable cations include, but are not limited to, sodium, potassium, calcium, aluminum, lithium, and ammonium.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

As defined herein, “pharmaceutically acceptable salts thereof” include salts of at least one compound of Formulas I, II (including II-1, II-2 or II-3) or III, and salts of the stereoisomers of at least one compound of Formulas I, II (including II-1, II-2 or II-3) or III, such as salts of enantiomers, and/or salts of diastereomers.

“Treating”, “treat”, or “treatment” or “alleviation” refers to administering at least one compound and/or at least one pharmaceutically acceptable salt thereof disclosed herein to a subject in recognized need thereof that has, for example, cancer disease, or has a symptom of, for example, cancer disease, or has a predisposition toward, for example, cancer disease, with the purpose to cure, heal, alleviate, relieve, alter, remedy, ameliorate, improve, or affect, for example, cancer disease, the symptoms of, for example, cancer disease, or the predisposition toward, for example, cancer disease.

The term “effective amount” refers to an amount of at least one compound, stereoisomers thereof, pharmaceutically acceptable salts thereof and solvates thereof, disclosed herein effective to “treat,” as defined above, a disease or disorder in a subject. In the case of cancer, the effective amount may cause any of the changes observable or measurable in a subject as described in the definition of “treating”, “treat”, “treatment” and “alleviation” above. For example, the effective amount can reduce the number of cancer or tumor cells; reduce the tumor size; inhibit or stop tumor cell infiltration into peripheral organs including, for example, the spread of tumor into soft tissue and bone; inhibit and stop tumor metastasis; inhibit and stop tumor growth; relieve to some extent one or more of the symptoms associated with the cancer, reduce morbidity and mortality; improve quality of life; or a combination of such effects. An effective amount may be an amount sufficient to decrease the symptoms of a disease responsive to inhibition of PARP. For cancer therapy, efficacy in vivo can, for example, be measured by assessing the duration of survival, time to disease progression (TTP), the response rates (RR), duration of response, and/or quality of life. Effective amounts may vary, as recognized by those skilled in the art, depending on route of administration, excipient usage, and co-usage with other agents.

The term “inhibition” indicates a decrease in the baseline activity of a biological activity or process. “Inhibition of PARP” refers to a decrease in the activity of PARP as a direct or indirect response to the presence of at least one compound and/or at least one pharmaceutically acceptable salt disclosed herein, relative to the activity of PARP in the absence of the at least one compound and/or the at least one pharmaceutically acceptable salt thereof. The decrease in activity is not bound by theory and may be due to the direct interaction of the at least one compound, stereoisomers thereof, and pharmaceutically acceptable salts thereof disclosed herein with PARP, or due to the interaction of the at least one compound and/or at least one pharmaceutically acceptable salt disclosed herein, with one or more other factors that in turn affect PARP activity. For example, the presence of at least one compound, stereoisomers thereof, and pharmaceutically acceptable salts thereof disclosed herein, may decrease PARP activity by directly binding to the PARP, by causing (directly or indirectly) another factor to decrease PARP activity, or by (directly or indirectly) decreasing the amount of PARP present in the cell or organism.

The term “prevention” refers to all of the actions in which a disease is restrained or the occurrence of a disease is retarded by the administration of the combination.

The term “delay of progression” refers to administration of the combination to patients being in a pre-stage or in an early phase, of the first manifestation or a relapse of the disease to be treated, in which a pre-form of the corresponding disease is diagnosed or which patients are in a condition during a medical treatment or a condition resulting from an accident, under which it is likely that a corresponding disease will develop.

The term “at least one substituent” disclosed herein includes, for example, from 1 to 4, such as from 1 to 3, further as 1 or 2, substituents. For example, “at least one substituent R¹²” disclosed herein includes from 1 to 4, such as from 1 to 3, further as 1 or 2, substituents selected from the list of R¹² as described herein.

The terms “administration”, “administering” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, rabbit) and most preferably a human.

The terms “cancer” or “tumor” herein mean or describe the physiological condition involving abnormal cell growth with the potential to invade or spread to other parts of the body. The “disease” refers to any disease, discomfort, illness, symptoms or indications, and can be substituted with the term “disorder” or “condition”.

In some embodiments, the cancer is solid cancers including, but not limited to, colorectal cancer, gastric cancer, small cell lung cancer, breast cancer, ovarian cancer, fallopian tube carcinoma, peritoneal carcinoma, melanoma, glioblastoma or lymphoma. In some embodiments, the cancer is glioblastoma with unmethylated MGMT promoter. In some embodiments, the cancer is recurrent/refractory glioblastoma.

### PARP Inhibitors

“PARP inhibitor” means a compound of Formula (I), or a stereoisomer thereof, a pharmaceutically acceptable salt thereof, or a solvate thereof.

As disclosed in each of the above five aspects, the PARP inhibitor is a compound of Formula (I),

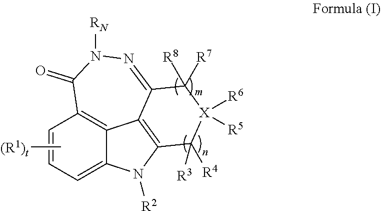

a stereoisomer thereof, a pharmaceutically acceptable salts thereof, or a solvate thereof, wherein:
R_{N} is selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
X is selected from the group consisting of C, N, O, and S;
m and n, which may be the same or different, are each an integer of f 0, 1, 2, or 3; t is an integer of 0, 1, 2, or 3;
R¹, at each occurrence, is independently selected from halogen, CN, NO₂, OR⁹, NR⁹R¹⁰, NR⁹COR¹⁰, NR⁹SO₂R¹⁰, CONR⁹R¹⁰, COOR⁹, SO₂R⁹, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R² is selected from hydrogen, COR⁹, CONR⁹R¹⁰, CO₂R⁹, SO₂R⁹, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R³, R⁴, R⁵, R⁶, R⁷ and R⁸, which may be the same or different, are each independently selected from hydrogen, halogen, —NR⁹R¹⁰, —OR⁹, oxo, —COR⁹, —CO₂R⁹, —CONR⁹R¹⁰, —NR⁹CONR¹⁰R¹¹, —NR⁹CO₂R¹⁰, —NR⁹SO₂R¹⁰, —SO₂R⁹, alkyl, alkenyl, cycloalkyl, aryl, heterocyclyl, alkynyl, and heteroaryl, wherein each of the alkyl, alkenyl, alkynyl, cycloalkyl, aryl, heterocyclyl, and heteroaryl is independently optionally substituted with at least one substituent R¹², or (R³ and R⁴), and/or (R⁴ and R⁵), and/or (R⁵ and R⁶), and/or (R⁶ and R⁷), and/or (R⁷ and R⁸), together with the atom(s) they are attached, form a 3- to 8-membered saturated, partially or fully unsaturated ring having 0, 1 or 2 heteroatoms independently selected from —NR¹³—, —O—, —S—, —SO—or —SO₂—, and said ring is optionally substituted with at leaset one substituent R¹², provided that

when X is O, R⁵ and R⁶ are absent,

when X is N, R⁶ is absent, an

when X is S, R⁵ and R⁶ are absent, or at least one of R⁵ and R⁶ is oxo,

when one of R³ and R⁴ is oxo, the other is absent,

when one of R⁷ and R⁸ is oxo, the other is absent, and

when X is C and one of R⁵ and R⁶ is oxo, the other is absent;

R⁹, R¹⁰, and R¹¹, which may be the same or different, are each selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R¹² is selected from CN, halogen, haloalkyl, NO₂, —NR′R″, —OR′, oxo, —COR′, —CO₂R′, —CONR′R″, —NR′CONR″R′″, —NR′CO₂R″, —NR′SO₂R″, —SO₂R′, alkyl, alkenyl, alkynyl, cycloalkyl, heterocyclyl, aryl, and heteroaryl, wherein R′, R″, and R′″ are independently selected from hydrogen, haloalkyl, alkyl, arylalkyl, alkenyl, alkynyl, cycloalkyl, heterocyclyl, aryl, and heteroaryl, or (R′ and R″), and/or (R″ and R′″) together with the atoms to which they are attached, form a 3- to 8-membered saturated, partially or fully unsaturated ring having 0, 1 or 2 additional heteroatoms independently selected from —NR¹³—, —O—, —S—, —SO— and —SO₂—,
R¹³ is selected from hydrogen, alkyl, cycloalkyl, aryl, heteroaryl, and heterocyclyl.

In some embodiments, the PARP inhibitor is a compound of Formula (II),

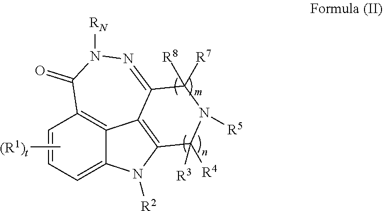

a stereoisomer thereof, a pharmaceutically acceptable salts thereof, or a solvate thereof, wherein:
R_{N} is selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
m and n, which may be the same or different, are each an integer of 0, 1, 2, or 3; t is an integer of 0, 1, 2, or 3;
R¹, at each occurrence, is independently selected from halogen, CN, NO₂, OR⁹, NR⁹R¹⁰, NR⁹COR¹⁰, NR⁹SO₂R¹⁰, CONR⁹R¹⁰, COOR⁹, SO₂R⁹, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R² is selected from hydrogen, COR⁹, CONR⁹R¹⁰, CO₂R⁹, SO₂R⁹, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R³, R⁴, R⁵, R⁷ and R⁸, which may be the same or different, are each independently selected from hydrogen, halogen, —NR⁹R¹⁰, —OR⁹, oxo, —COR⁹, —CO₂R⁹, —CONR⁹R¹⁰, —NR⁹CONR¹⁰R¹¹, —NR⁹CO₂R¹⁰, —NR⁹SO₂R¹⁰, —SO₂R⁹, alkyl, alkenyl, cycloalkyl, aryl, heterocyclyl, alkynyl, and heteroaryl, wherein each of the alkyl, alkenyl, alkynyl, cycloalkyl, aryl, heterocyclyl, and heteroaryl is independently optionally substituted with at least one substituent R¹², or (R³ and R⁴), and/or (R⁴ and R⁵), and/or (R⁵ and R⁷), and/or (R⁷ and R⁸), together with the atom(s) they are attached, form a 3- to 8-membered saturated, partially or fully unsaturated ring having 0, 1 or 2 heteroatoms independently selected from —NR¹³—, —O—, —S—, —SO—, and —SO₂—, and said ring is optionally substituted with at leaset one substituent R¹², provided that

when one of R³ and R⁴ is oxo, the other is absent, and when one of R⁷ and R⁸ is oxo, the other is absent; R⁹, R¹⁰, and R¹¹ , which may be the same or different, are each selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;

R¹² is selected from CN, halogen, haloalkyl, NO₂, —NR′R″, —OR′, oxo, —COR′, —CO₂R′, —CONR′R″, —NR′CONR″R′″, —NR′CO₂R″, —NR′SO₂R″, —SO₂R′, alkyl, alkenyl, alkynyl, cycloalkyl, heterocyclyl, aryl, and heteroaryl, wherein R′, R″, and R′″ are independently selected from hydrogen, haloalkyl, alkyl, arylalkyl, alkenyl, alkynyl, cycloalkyl, heterocyclyl, aryl, and heteroaryl, or (R′ and R″), and/or (R″ and R′″) together with the atoms to which they are attached, form a 3- to 8-membered saturated, partially or fully unsaturated ring having 0, 1 or 2 additional heteroatoms independently selected from —NR¹³—, —O—, —S—, —SO— or —SO₂—; and
R¹³ is selected from hydrogen, alkyl, cycloalkyl, aryl, heteroaryl, and heterocyclyl.

In some embodiments, the PARP inhibitor is selected from the compound the following compounds,

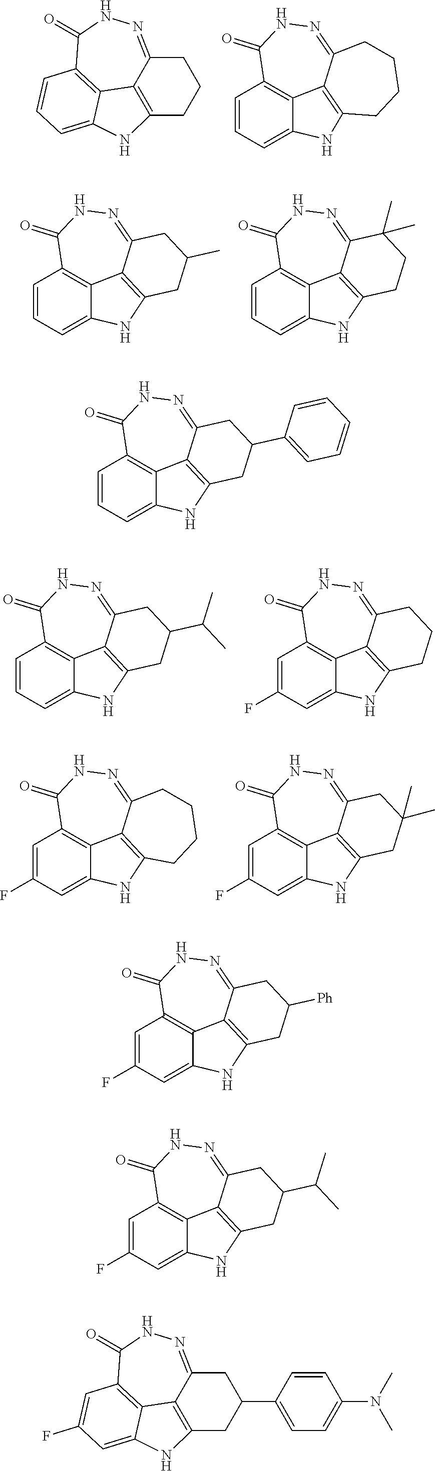

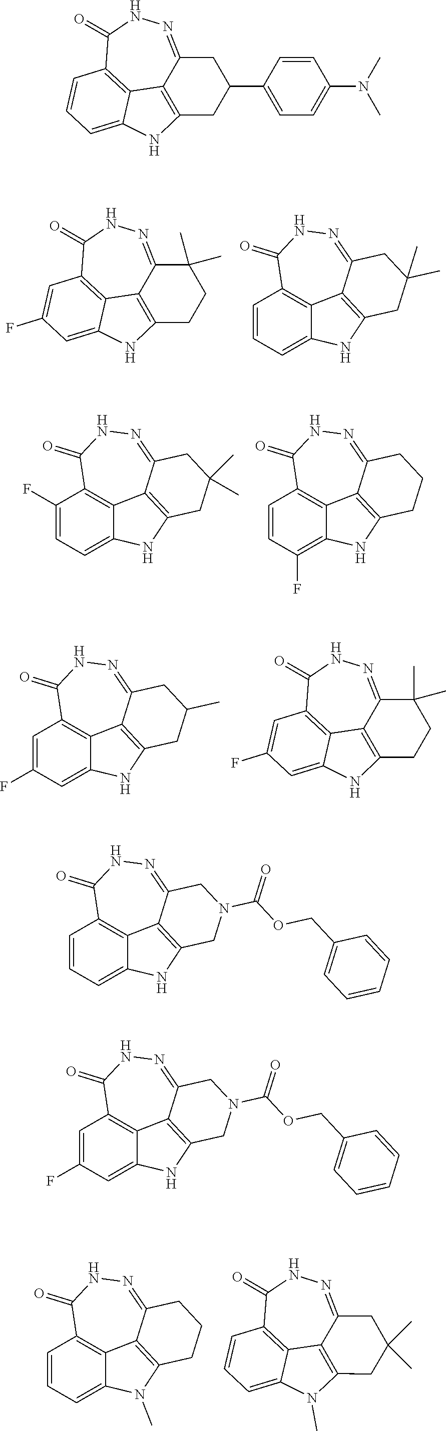

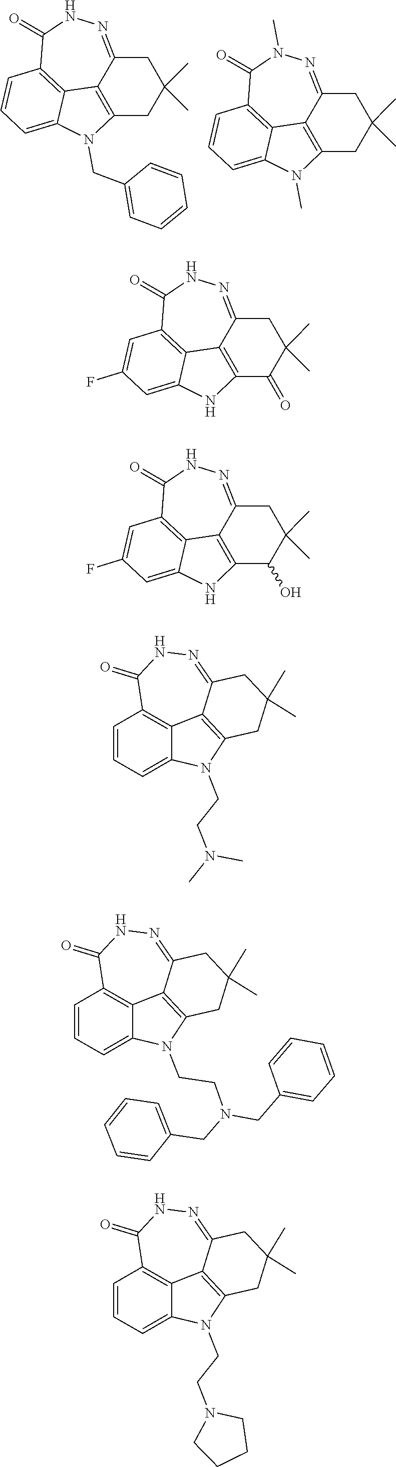

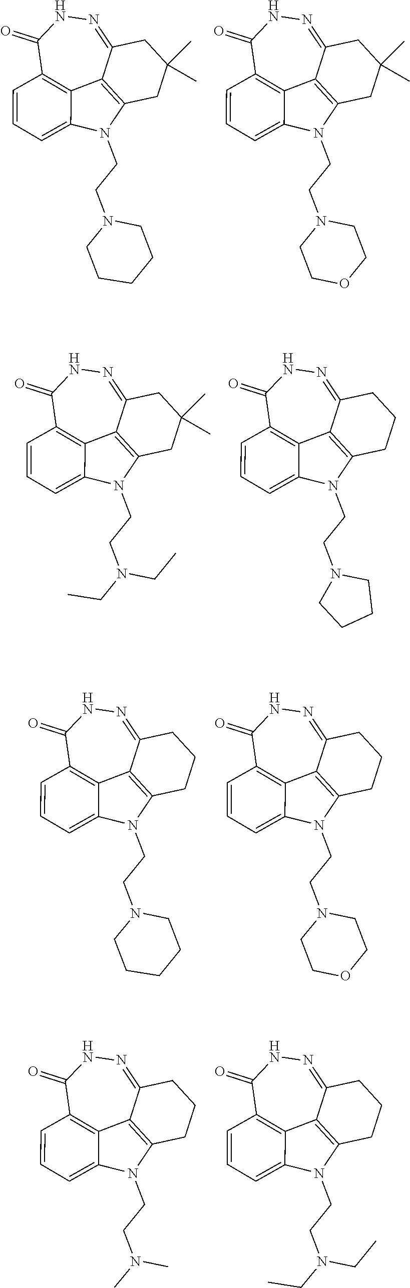

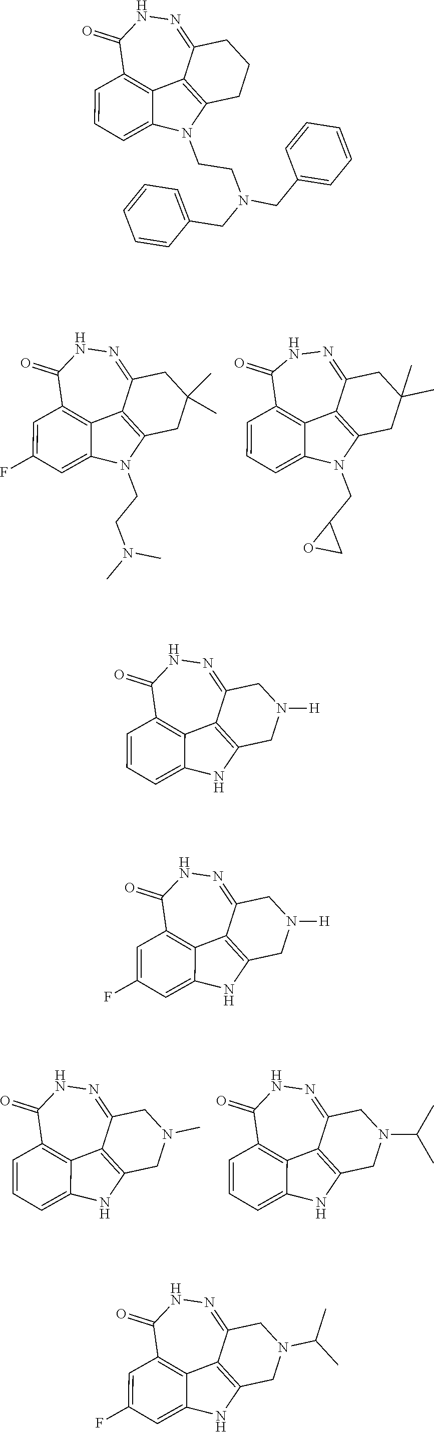

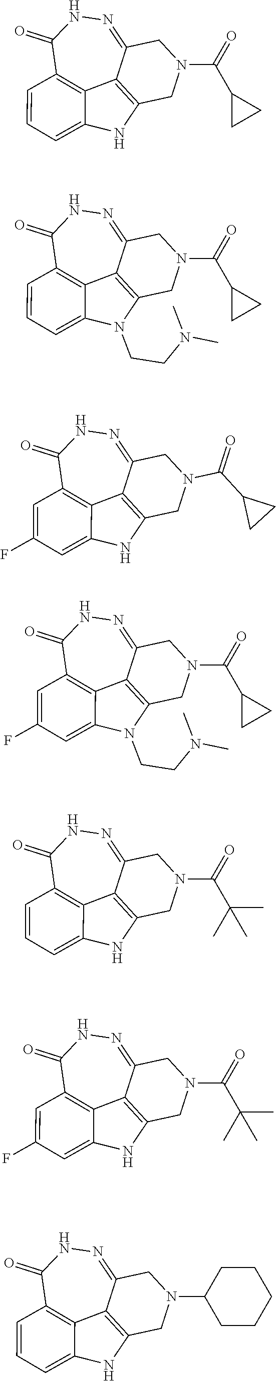

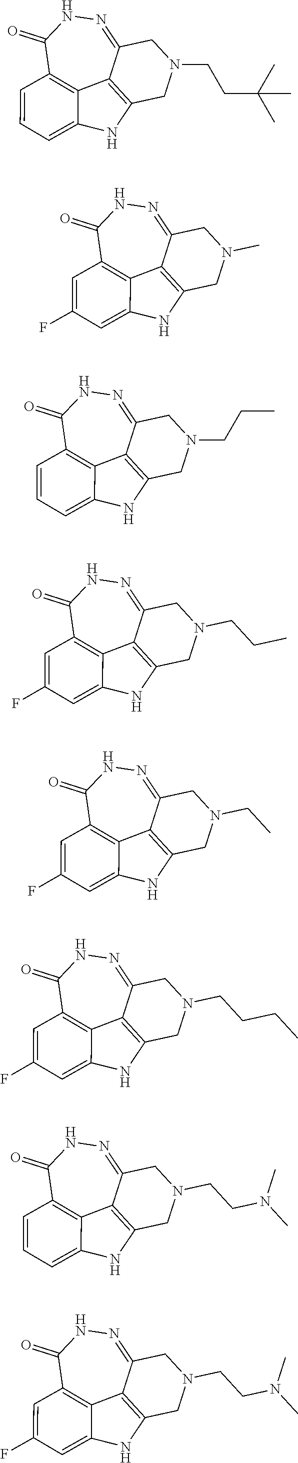

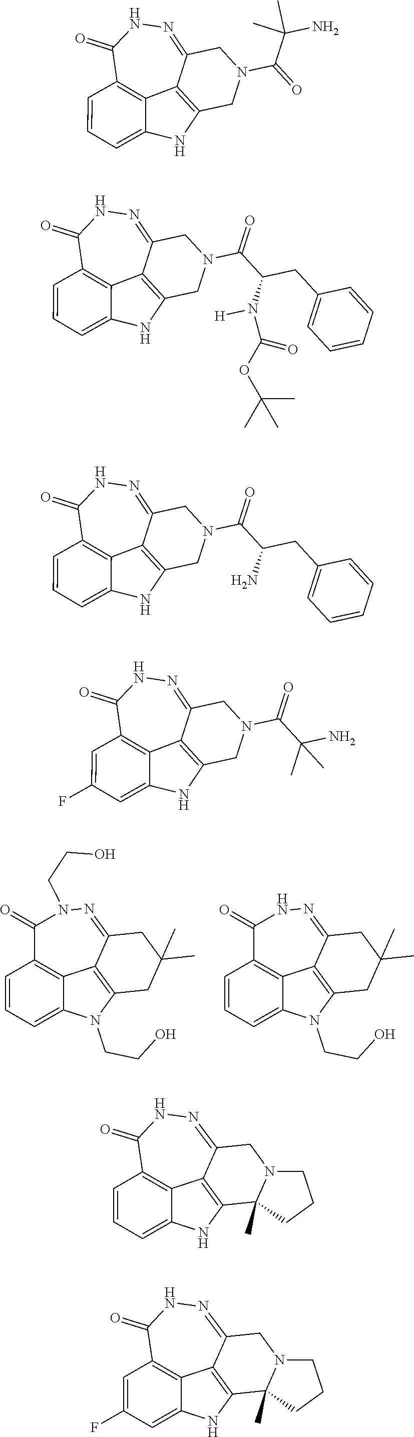

a stereoisomer thereof, a pharmaceutically acceptable salts thereof, or a solvate thereof.

As disclosed in each of the above five aspects, the PARP inhibitor is a compound of Formula (III)—i.e., Compound A,

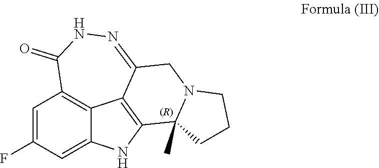

or a pharmaceutically acceptable salt thereof.

As disclosed in each of the above five aspects, the PARP inhibitor is a compound of Formula (IV)—i.e., Compound B.

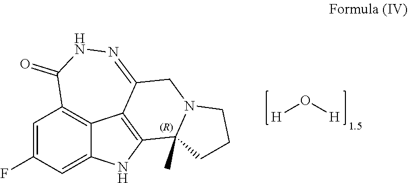

The PARP inhibitor disclosed herein, such as the compound of Formula (III) and (IV), may be synthesized by synthetic routes disclosed in WO 2013/097225A1 and WO 2017032289A, the entire disclosure of which is expressly incorporated herein by reference.

### Combination Therapy

The combination therapy may be administered as a simultaneous, or separate or sequential regimen. When administered sequentially, the combination may be administered in two or more administrations. The combined administration includes co-administration, using separate formulation, and consecutive administration in either order, wherein preferably there is a time period while both (or all) active agents simultaneously exert their biological activities.

Suitable dosages for any of the above co-administered agents are those presently used and may be lowered due to the combined action (synergy) of the PARP inhibitor and temozolomide and/or radiation therapy, such as to increase the therapeutic index or mitigate toxicity or other side-effects or consequences.

In a particular embodiment of anti-cancer therapy, the PARP inhibitor and temozolomide and/or radiation therapy may be further combined with surgical therapy.

In an embodiment of each of the above five aspects, the amounts of the PARP inhibitor and temozolomide and/or radiation therapy disclosed herein and the relative timings of administration be determined by the individual needs of the patient to be treated, administration route, severity of disease or illness, dosing schedule, as well as evaluation and judgment of the designated doctor.

For example, the administered dosage of the PARP inhibitor is 1-120 mg or 1-80 mg or 1-60 mg or 1-50 mg or 1-40 mg or 1-30 mg or 1-20 mg or 1-10 mg or 20-80 mg or 20-60 mg or 20-50 mg or 20-40 mg or 20-30 mg (in terms of the parent compound), and the administration frequency is once to twice a day; preferably, the administered dosage of the PARP inhibitor is 1-80 mg (in terms of the parent compound), and the administration frequency is twice a day (BID). In some cases, it is more suitable to apply the lower end of the above described dosage ranges, while in other cases the higher dosages may be used without causing harmful side effects.

The PARP inhibitor and temozolomide disclosed herein may be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

In one embodiment of each of the above aspect, the PARP inhibitor and temozolomide disclosed herein may be administered in different route. In a preferred embodiment, the PARP inhibitor is administered orally, and temozolomide is administered orally.

The dosage of temozolomide for practicing the combination therapy is 10 to 400 mg per m² of the patient's body surface area per day, more preferably 10 to 150 mg/m² and most preferably 20-120 mg/m²/day; or 20-75 mg/m²/day. It is preferred that the daily dosage of temozolomide be administered once per day for a 2 to 10 day period, more preferably for a 3 to 8 day period and most preferably for a 5 day period. The temozolomide dosing periods may be repeated in cycles of 28 to 42 days, more preferably 28 to 35 days, and most preferably 28 days. That is, 28 to 42 days after the first day of temozolomide administration, another temozolomide administration period may be started.

Alternatively the temozolomide may be administered for a much longer period at reduced dosage. For example, the temozolomide may be administered daily for 11 days to six weeks at a dosage of 20 to 120 mg/m²/day. Temozolomide may be administered orally in capsule form wherein it is admixed with conventional pharmaceutical carriers.

In an embodiment above aspects, temozolomide is administrated to a subject at a dose of 20-120 QD and the PARP inhibitor Compound A or Compound B is administrated to a subject at a dose of 1-120 mg BID.

### EXAMPLES

The present invention is further exemplified, but not limited, by the following examples that illustrate the invention. In the examples of the present invention, the techniques or methods, unless expressly stated otherwise, are conventional techniques or methods in the art.

### Example 1. Preparation of Compound A and Compound B

Step 1: Synthesis of Compound-2

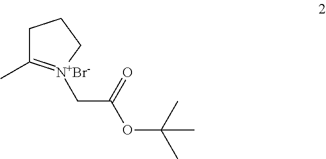

t-Butyl bromoacetate (51.7 Kg) was dissolved in anhydrous acetonitrile (72 Kg). The temperature was raised to 65-75° C., then methyl pyrroline (22 Kg) was added. The reaction mixture was condensed after the reaction was completed, the residual acetonitrile was removed by adding THF and then condensing. After GC showed a complete removal of acetonitrile, more THF was added and stirred. The resulting solid was filtered and collected. 44.1 Kg of off white solid Compound-2 was obtained. ¹H NMR (400 MHz, DMSO-d6) δ 4.91 (s, 2H), 4.15 (m, 2H), 3.29 (m, 2H), 2.46 (s, 3H),), 2.14 (m, 2H), 1.46 (s, 9H) ppm.

Step 2: Synthesis of Compound-3

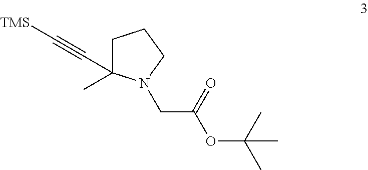 [SMILES: CC(C)(C)OC(=O)CN1CCCC1(C)C#C[Si](C)(C)C]

To a cool (−60° C.) solution of trimethylsilyl acetyne (12.4 Kg) in THF was added a solution of n-butyl lithium in hexane (43.4 Kg). After complete addition of n-butyl lithium solution, the resulting mixture was stirred for additional 1-2 h and then the entire solution was transferred into a suspension of Compound-2 (31 Kg) in THF cooled at −60° C. After transfer completion, the resulting mixture was warmed to room temperature and stirred for 1 h. The reaction was quenched with water, extracted with petroleum. The organic phase was washed with brine, dried over sodium sulfate, condensed to give 25.1 Kg of Compound-3. ¹H NMR (400 MHz, DMSO-d6) δ 3.34 (d, J=16.0 Hz, 1H), 3.15 (m, 1H), 2.78 (d, J=16.0 Hz, 1H), 2.27 (m, 1H), 1.93 (m, 1H), 1.68 (m, 3H), 1.41 (s, 9H), 1.24 (s, 3H), 0.13 (s, 9 H) ppm.

Step 3: Synthesis of Compound-4

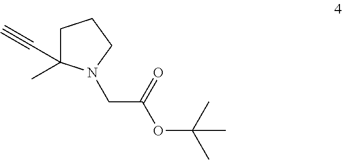 [SMILES: C#CC1(C)CCCN1CC(=O)OC(C)(C)C]

To a cool (0-5° C.) solution of 70.1 Kg of Compound-3 in THF was added tetrabutylammonium fluoride (13.3 Kg) in THF. After de-silylation was completed, the reaction was quenched with water, extracted with petroleum (290 Kg) and the organic phase was condensed and passed through a pad of silica gel. The filtrate was condensed to give 48 Kg of Compound-4. ¹H NMR (400 MHz, DMSO-d6) δ 3.36 (d, J=16.0 Hz, 1H), 3.15 (m, 1H), 2.82 (d, J=16.0 Hz, 1H), 2.28 (m, 1H), 1.97 (m, 1H), 1.70 (m, 3H), 1.41 (s, 9H), 1.26 (s, 3H) ppm.

Step 4: Syntheses of Compound-5

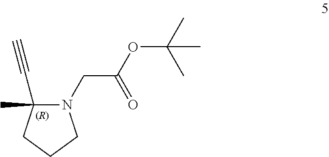

A solution of Compound-4 (48 Kg) in THF was warmed to 50-60° C. To the above solution was added a solution of (−)-di-p-methylbenzoyl-L-tartaric acid (69.6 Kg) in THF. The resulting mixture was stirred at 50-60° C. 1-2 h and then gradually cooled to 0-10° C. The resulting salt solid was filtered and re-suspended in methyl tert-butyl ether and heated at 50-60° C. for 1 h. The mixture was gradually cooled to 0-5° C. The resulting solid was filtered to give 13.1 Kg of off-white solid. The solid was treated with aqueous sodium hydroxide, extracted with petroleum, condensed to give 13.1 Kg of Compound-5 (ee≥96%). ¹H NMR (400 MHz, DMSO-d6) δ 3.36 (d, J=16.0 Hz, 1H), 3.15 (m, 1H), 2.82 (d, J=16.0 Hz, 1H), 2.29 (m, 1H), 1.97 (m, 1H), 1.70 (m, 3H), 1.41 (s, 9H), 1.26 (s, 3H) ppm.

Step 5: Syntheses of Compound-6

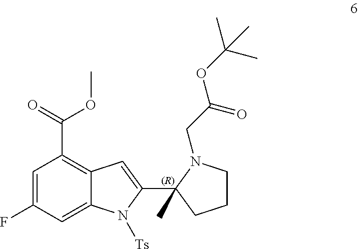

Intermediate B (14 Kg), bis(triphenyl)palladium dichloride (0.7 Kg), CuI (0.42 Kg) and tetramethyl guanidine (11.5 Kg) were dissolved in DMF (48.1 Kg). The resulting solution was stirred and de-gassed and then heated under nitrogen. A solution of Compound-5 (9.24 Kg) in DMF (16 Kg) was added dropwise. After coupling, the organic phase was condensed, the resiue was stirred with water (145 Kg) and methyl t-butyl ether (104 Kg), the entire mixture passed trough a pad of celite, separated. The organic phase was washed with a solution of thiourea (14 Kg) in water (165 kg) and brine (100 Kg), condensed. The residue was dissolved in a mixture of n-heptane (120 Kg) and ethyl acetate (28 Kg). The solution was mixed with charcoal (1.4 kg), heated at 40-50° C. for 1-2 h, fltered though a pad of silica gel. The filtrate was condensed to give Compound-6 solid (14.89 Kg) and the liquid filtrate (13 Kg heptane solution, contains 1.24 Kg of Compound-6). ¹H NMR (400 MHz, DMSO-d6) δ 7.85 (d, J=9.6 Hz, 1H), 7.55 (m, 3H), 7.32 (m, 2H), 3.87 (s, 3H), 3.37 (d, J=16.0 Hz, 1H), 3.22 (m, 1H), 2.94 (d, J=16.0, Hz, 1H), 2.60 (m, 1H), 2.48 (m, 1H), 2.29 (s, 3h), 2.26 (m, 1 H), 1.82 (m, 2H), 1.49 (s, 3H), 1.43 (s, 9H) ppm.

Step 6: Syntheses of Compound-7

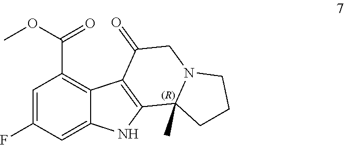

The above heptane solution of Compound-6 was added into a cold trifluoromethane sulfonic acid (66.1 Kg) while maintaining the internal temperature below 25° C. Then solid Compound-6 (14.87 Kg) was added batchwise. After complete addition of Compound-6, the reaction mixture was warmed to 25-30° C. and stiired until the reaction was completed. The entire mixture was poured into a solution of sodium acetate (123.5 Kg) in water (240 Kg). pH of the solution was then adjusted to 7-8 by adding solid potassium carbonate (46.1 Kg). The mixture was extracted wuth dichloromethane (509 Kg), condensed. The residue was mixed with n-heptane (41 Kg), condensed again to give the precipitate which was filtered and washed by n-heptane (8 Kg) and dried. 8.78 Kg of Compound-7 was obtained. ¹H NMR (400 MHz, DMSO-d6) δ 12.30 (s, 1H), 7.35 (dd, J=9.2, 1.6 Hz, 1H), 7.08 (dd, J=9.2, 1.6 Hz, 1H), 3.79 (s, 3H), 3.68 (d, J=17.2 Hz, 1H), 3.21 (d, J=17.2 Hz, 1H), 3.06 (m, 1H), 2.68 (m, 1H), 1.96 (m, 1H), 1.74 (m, 1H), 1.49 (s, 3H) ppm.

Step 7: Syntheses of Compound A-Crude 1

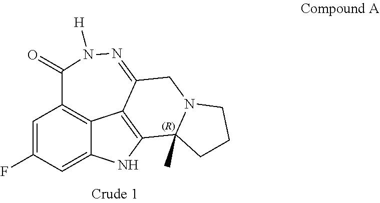

Compound-7 (8.76 Kg) was dissolved in methanol (69 Kg) and internally cooled below 25° C. Acetic acid (9.3 Kg) and hydrazine hydrate (7.4 Kg, 85%) were added while maintaining internal temperature below 25° C. After de-gassed and re-filled with nitrogen (repeated three times), the reaction mixture was stirred at 55-60° C. for 4 h. After a complete reaction, the mixture was mixed with water (29 Kg). The organic phase was condensed and potassium carbonate (12.5 Kg) in water (40 Kg) was added. The resulting solid was filtered, washed with water (18.3 Kg). The solid was slurred with water (110 Kg), centrifuged, dried and slurred with ethanol (9.4 Kg), centrifuged, filtered, washed with ethanol, dried in vaccum to give Compound A-Crude 1 (7.91 Kg). ¹H-NMR (600 MHz, DMSO-d6) δ 12.0 (s, 1H), 10.2 (s, 1H), 7.31 (dd, 1H, J=9.6, 2.0 Hz), 7.19 (dd, 1H, J=9.6, 2.0 Hz), 3.77 (d, 1H, J=16.4 Hz), 3.34 (d,1H, J=16.4 Hz), 2.97-3.02 (m, 1H), 2.54-2.58 (m, 1H), 2.35-2.40 (m, 1H), 1.90-1.94 (m, 1H), 1.73-1.75 (m, 1H), 1.47 (s, 3H), 1.43-1.45 (m, 1H) ppm. MS (ESI) m/e [M+1]⁺299.

Step 8: Synthesis of Compound A-Crude 2

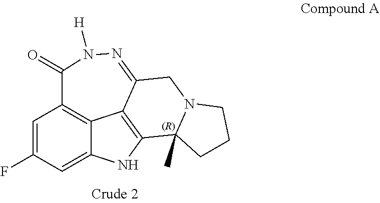

Under nitrogen protection, Compound A (Crude 1) (7.88 Kg) was stirred with isopropanol (422 Kg) and heated at 70-80° C. for 1-2 h until the solid disappeared completely. A solution of (+)-di-p-methylbenzoyl-D-tartaric acid (10.25 Kg) in isopropanol (84.4 Kg) was added. The mixture was stirred for 14-16 h, filtered and washed with isopropanol (16 Kg), dried. The resulting salt was added into a stirred solution of potassium carbonate (6.15 Kg) in water (118 Kg). The precipitate was centrifuged, filtered, washed with water (18 Kg). The solid was slurred with water (110 Kg), centrifuged, dried. The solid was dissolved in THF (75 Kg), active carbon (0.8 Kg) was added. The mixture was degassed and re-protected by nitrogen, stirred and heated at 40-45° C. for 1-2 h, cooled, filtered through celite, condensed to give the solid which was further slurred with ethanol (6.5 Kg), filtered to give 5.6 Kg of Compound A crude 2. ¹H NMR (400 MHz, DMSO-d6) δ 12.0 (s, 1H), 10.2 (s, 1H), 7.31 (dd, 1H, J=9.6, 2.0 Hz), 7.19 (dd, 1H, J=9.6, 2.0 Hz), 3.77 (d, 1H, J=16.4 Hz), 3.34 (d,1H, J=16.4 Hz), 2.97-3.02 (m, 1H), 2.54-2.58 (m, 1H), 2.35-2.40 (m, 1H), 1.90-1.94 (m, 1H), 1.73-1.75 (m, 1H), 1.47 (s, 3H), 1.43-1.45 (m, 1H) ppm. MS (ESI) m/e [M+1]⁺299.

Step 9: Synthesis of Compound B

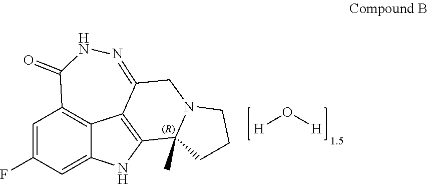

Compound A-Crude 2 (5.3 Kg) was mixed with a solution of isopropanol (41.6 Kg) and water (15.9 Kg). The mixture was degassed and re-protected under nitrogen and then heated to 60° C. and stirred for 2-4 h until the solid was dissolved completely. The temperature was raised to 70-80° C. and water (143 Kg) was added. The resulting mixture was heated to the internal temperature of 70-80° C. and then the heating was stopped but stirred gently for 16 h. The precipitate was filtered, washed with water (19 Kg) and slurred with water (21 kg) for 2 h. The resulting solid was filtered, washed with water (20 Kg). The filtered solid was dried at the temperature below 45° C. for 24-36 h. Compound A sesqui-hydrate (4.22 kg) was obtained with particle sizes of D90=51.51 um, D50=18.62 um, D10=7.63 um. This range of PSD is almost ideal for formulation development.

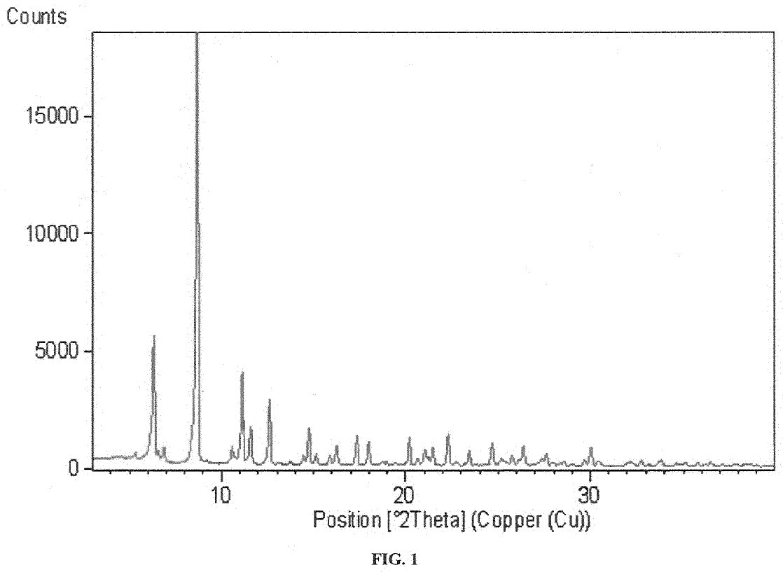

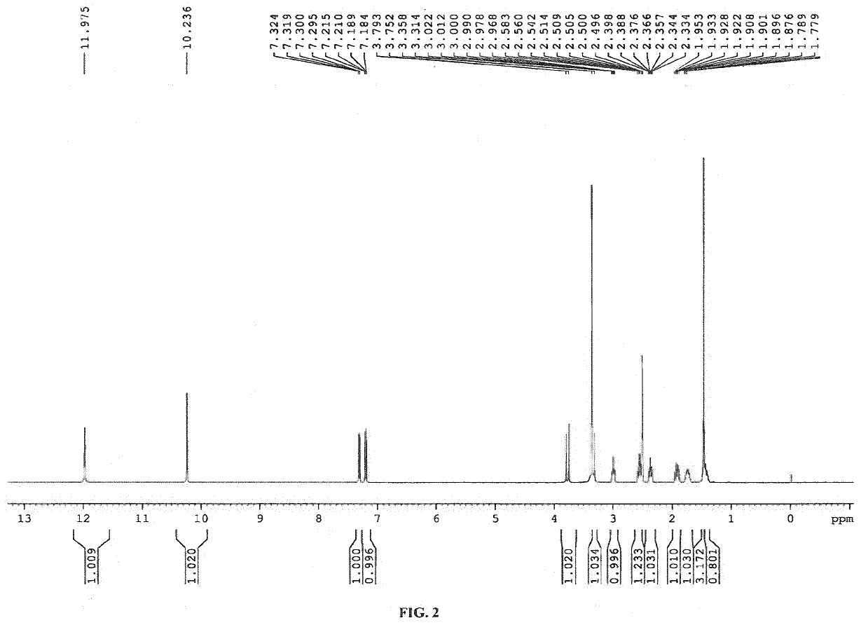

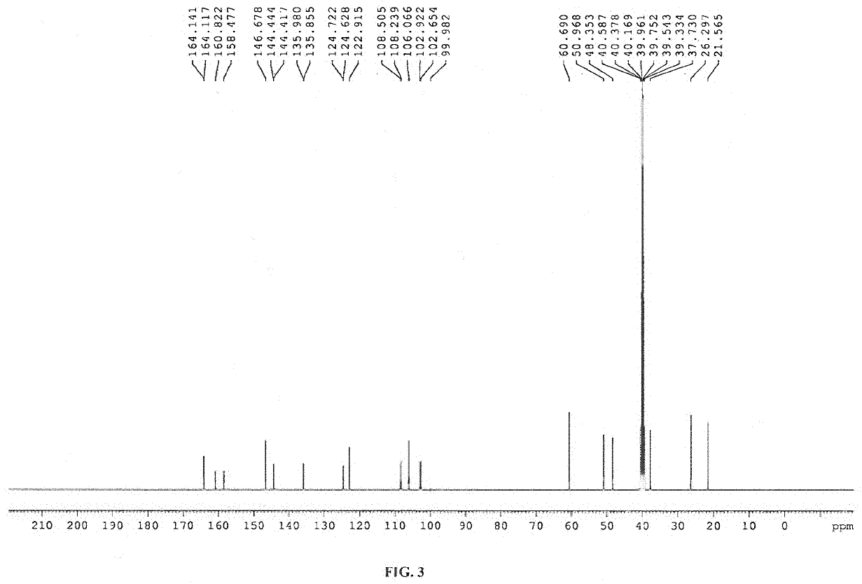

The powder X-ray diffraction pattern (PXRD) was used to characterize Crystal Compound B, see FIG. 1. ¹H-NMR for Crystal Compound B, is shown in FIG. 2. ¹³C-NMR for Crystal Compound B is shown in, see FIG. 3.

### Example 2 Effect of the Combination of PARP Inhibitor and Temozolomide (TMZ)

Compound B as a single agent has demonstrated excellent in vitro activity against tumor cell lines with defects of the HR pathway. In vivo, Compound B showed strong anti-tumor activity against a BRCA1-mutant mouse xenograft model (MDA-MB-436 breast cancer) and was 16-fold more potent than olaparib. In a pharmacokinetic (PK)/pharmacodynamic (PD) study, oral administration of Compound B resulted in time- and dose-dependent inhibition of PARylation in MDA-MB-436 breast cancer xenografts in mice. Inhibition of PARylation in the tumor tissues correlated well with tumor drug concentrations of Compound B.

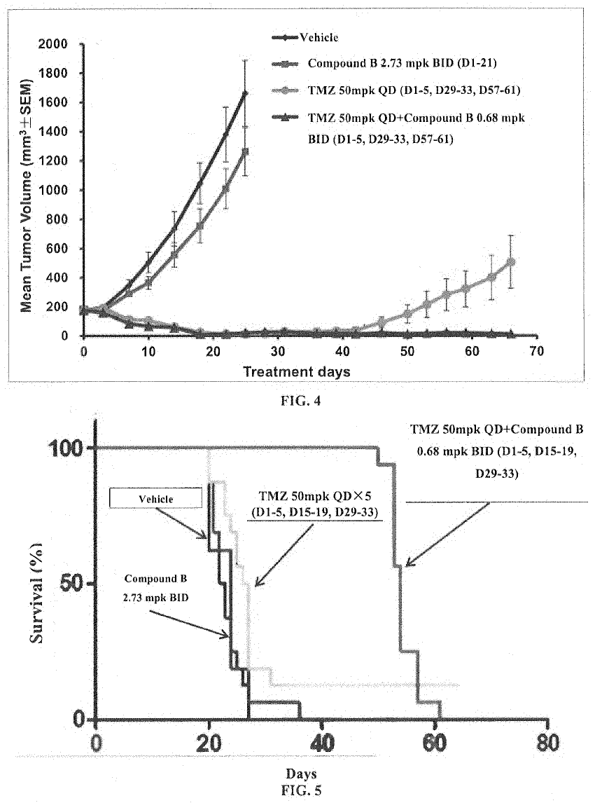

The anti-proliferative effect of Compound B in combination with TMZ was evaluated in 8 human GB cell lines resistant to single-agent TMZ (EC50 of 32 M or greater). In 7 of 8 cell lines, Compound B demonstrated synergism with TMZ with a shift in EC50 for TMZ of 5-fold or greater. This synergism was also demonstrated in vivo in an H209 small cell lung cancer xenograft model (FIG. 4). Compound B (2.73 mg/kg BID×21 days) as single-agent treatment had no significant effect on tumor growth. TMZ (50 mg/kg QD, Days 1-5 of each 28-day cycle) as single-agent treatment was quite effective in this model resulting in objective responses in all animals (1 PR and 7 CRs in 8 animals) after the first cycle of treatment. However, 6 of these 8 animals developed TMZ resistance after three cycles of treatment, and the mean tumor volume reached 505 mm³ on Day 66. Addition of Compound B (0.68 mg/kg BID, Days 1-5 of each 28-day cycle) resulted in objective responses in all animals (2 PRs and 6 CRs in 8 animals) after the first cycle of treatment. After completion of 3 cycles of treatment (on Day 66), most animals were still tumor-free (6/8), and the mean tumor volume was 12 mm³. Thus, the combination of Compound B and TMZ significantly enhanced TMZ anti-tumor activity and delayed resistance.

Given the significant brain penetrance of Compound B, its activity was further explored in an intracranial tumor model in nude mice for H209-T small cell lung cancer xenografts (FIG. 5). H209-T is a TMZ-resistant cell line generated by treating H209-xenografted tumors with multiple cycles of TMZ in vivo. In this model, Compound B (2.73 mg/kg BID) as single-agent treatment had no significant effect on tumor growth, with a median survival of 24 days compared to median survival of 22.5 days in the vehicle-treated group. H209-T intracranial xenografts showed resistance to the TMZ treatment alone (50 mg/kg), with median survival of 26.5 days. However, the combination of Compound B and TMZ significantly prolonged animal survival compared to TMZ (p<0.01), with median survival of 54 days. The result suggests Compound B in combination with TMZ can overcome TMZ resistance in this intracranial model.

### Example 3: Clinical Trials

An open-label, multi-center, multiple-dose, dose-escalation Phase 1b/2 study of Compound B in combination with radiation therapy (RT) and/or temozolomide (TMZ) was conducted.

(1) Compound B Combined with RT in Patients with First-Line Glioblastoma (GB) with Unmethylated MGMT Promoter (‘Unmethylated GB’).

Compound B (60 mg BID) at increasing exposures of 2, 4, and 6 weeks in combination with RT was administrated to the patients for 6 to 7 weeks. After RT was completed, the patients received no further treatment.

(2) Compound B Combined with Both TMZ and RT in Subjects with First-Line Unmethylated GB.

Compound B (60 mg BID) in combination with RT for 6 to 7 weeks and increasing doses of TMZ was administrated to the patients. After RT was completed, the patients received no further treatment.

(3) Compound B Combined with TMZ in Subjects with Recurrent/Refractory GB with Methylated or Unmethylated MGMT Promoter.

Compound B (60 mg BID) in combination with increasing doses of TMZ was administered to the patients on Days 1 to 21 of each 28-day cycle.

In the combination therapies (1), (2) and (3): Compound B was administered at the doses of 60 mg BID (PO); RT was administered QD×5 days/week for 6 to 7 weeks with 1.8 to 2 Gy/fraction for a total dose of up to 60 Gy; Flat-dosing was used for TMZ, and the first dose level of (20 mg) 40 mg QD corresponds to 23 mg/m² assuming an average body surface area of 1.73 m², subsequent dose levels of 80 mg and 120 mg correspond to 46 mg/m² and 69 mg/m², respectively are administered PO QD.

The results showed that all the combinations were safe and well tolerated.

### Example 4: Compound B+Temozolomide (TMZ) in Patients (pts) with Locally Advanced or Metastatic Solid Tumors

### Methods

This dose-escalation/expansion study is enrolling pts using a modified 3+3 design to establish the safety and MTD of Compound B plus TMZ. During dose escalation, pts receive Compound B plus escalating doses of TMZ QD on Days 1-7 (Arm A) or continuously (Arm B) of each 28-day cycle. In Arm A and Arm B: Compound B was administered at the doses of 60 mg PO BID; TMZ was administered at 20 mg, 40 mg, 80 mg, 120 mg QD corresponds to 12 mg/m², 23 mg/m², 46 mg/m²and 69 mg/m²assuming an average body surface area of 1.73 m², respectively will be administered PO QD.

The primary endpoint is safety/tolerability, including estimation of MTD and RP2D. Key secondary endpoints are PK profiles of TMZ and Compound B and antitumor activity (RECIST v1.1) of combination treatment; biomarker (eg, gBRCA) assessment is exploratory.

### Results

16 pts (Arm A, n=4, 40 mg TMZ; n=4, 80 mg TMZ; n=3, 120 mg TMZ; Arm B, n=4, 20 mg TMZ; n=1, 40 mg TMZ) with a median age of 69.5 yr (range 50-85) have enrolled; 8 remain on treatment. Prostate and small cell lung cancers (n=4 each) were the most common tumors; most pts (n=14) had received ≥3 prior treatments. Most common Compound B-related AEs were nausea (n=6), and nausea and thrombocytopenia (n=5 each) for TMZ. In Arm A, 2 pts at 120 mg TMZ reported a DLT of Grade 4 neutropenia >7 days. Neutropenia and thrombocytopenia (n=4 each) were ≥Grade 3 AEs occurring in >2 pts. No AE led to treatment discontinuation or death. Plasma exposure for Compound B and TMZ were consistent with single-agent trials. In the 7 pts with ≥1 post-baseline tumor assessment, 2 pts in Arm A (kidney, n=1; SCLC, n=1) achieved unconfirmed PRs. One pt with peritoneal cancer in Arm A had a 99.5% decrease in CA125 by wk 12 and SD for ˜25 wks.

### CONCLUSIONS

In pts with solid tumors, Compound B at the RP2D combined with pulsed or continuous flat dosed TMZ showed preliminary antitumor activity and a manageable safety profile with the expected toxicity of bone marrow suppression.

### Example 5: Compound B+Radiation Therapy (RT) and/or Temozolomide (TMZ) in Patients with Newly Diagnosed or Recurrent/Refractory Glioblastoma (GBM)

### Methods

This ongoing dose-escalation/expansion study was designed to determine the safety/tolerability and antitumor effects of Compound B (60 mg PO BID) in combination with RT and/or TMZ. The dose-escalation component consists of 3 arms:

(Arm A) Compound B (60 mg BID) at increasing exposures of 2, 4, or 6 weeks in combination with RT for 6 to 7 weeks, in newly diagnosed patients with GBM with unmethylated MGMT promoter (‘unmethylated GBM’)), and after RT is completed, subjects will receive no further study treatment;

(Arm B) Depending on the safety of the Arm A combination, Compound B in combination with RT for 6 to 7 weeks and increasing TMZ doses, in newly diagnosed patients with unmethylated GBM, and after RT is completed, subjects will receive no further study treatment;

(Arm C) Compound B in combination with increasing dose of TMZ administered on Days 1 to 21 of each 28-day cycle, in patients with recurrent/refractory methylated or unmethylated GBM.

In Arm A, Arm B and Arm C: Compound B was administered at the doses of 60 mg BID (PO); Radiation therapy (RT) was administered QD×5 days/week for 6 to 7 weeks with 1.8 to 2 Gy/fraction for a total dose of up to 60 Gy; flat-dosing was used for TMZ, and the first dose level of 40 mg QD corresponds to 23 mg/m² assuming an average body surface area of 1.73 m². Subsequent dose levels of 80 mg and 120 mg correspond to 46 mg/m² and 69 mg/m², respectively will be administered PO QD.

### Results

15 patients were enrolled (Arm A: 2-wk, n=3; 4-wk, n=6; Arm C: TMZ [40mg], n=6). One DLT (grade 3 nausea) was reported in Arm C. Across study arms, Compound B-related AEs occurring in ≥4 patients were nausea (n=6) and fatigue (n=5). Two patients experienced three Compound B-related AEs ≥grade 3 (diarrhea [Arm A: 4-wk, n=1]; fatigue and nausea [Arm C: n=1]). All three resolved with concomitant medication and treatment interruption (Arm A) or discontinuation (Arm C). Of the seven patients with ≥1 tumor assessment, one (Arm A: 4-wk) achieved an unconfirmed partial response; four (Arm A: 2-wk, n=2; 4-wk, n=2) had stable disease, and two patients (Arm A: 2-wk, n=1; Arm C: n=1) had progressive disease.

### CONCLUSIONS

Preliminary data from this ongoing study suggests Compound B in combination with RT and/or TMZ is generally well tolerated in patients with GBM.

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

The disclosures of all publications, patents, patent applications and published patent applications referred to herein by an identifying citation are hereby incorporated herein by reference in their entirety.

1. A method for the prevention, delay of progression or treatment of cancer in a subject in need thereof, comprising administering to the subject a therapeutically effective amount of a PARP inhibitor, in combination with a therapeutically effective amount of temozolomide and/or radiation therapy,
wherein the PARP inhibitor is a compound of Formula (I), 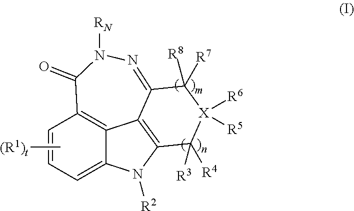
a stereoisomer thereof, a pharmaceutically acceptable salt thereof, or a solvate thereof, wherein:
R_{N} is selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
X is selected from the group consisting of C, N, O, and S;
m and n, which may be the same or different, are each an integer of 0, 1, 2, or 3;
t is an integer of 0, 1, 2, or 3;
R¹, at each occurrence, is independently selected from halogen, CN, NO₂, OR⁹, NR⁹R¹⁰, NR⁹COR¹⁰, NR⁹SO₂R¹⁰, CONR⁹R¹⁰, COOR⁹, SO₂R⁹, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R² is selected from hydrogen, COR⁹, CONR⁹R¹⁰, CO₂R⁹, SO₂R⁹, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R³, R⁴, R⁵, R⁶, R⁷ and R⁸, which may be the same or different, are each independently selected from hydrogen, halogen, —NR⁹R¹⁰, —OR⁹, oxo, —COR⁹, —CO₂R⁹, —CONR⁹R¹⁰, —NR⁹CONR¹⁰R¹¹, —NR⁹CO₂R¹⁰, —NR⁹SO₂R¹⁰, —SO₂R⁹, alkyl, alkenyl, cycloalkyl, aryl, heterocyclyl, alkynyl, and heteroaryl, wherein each of the alkyl, alkenyl, alkynyl, cycloalkyl, aryl, heterocyclyl, and heteroaryl is independently optionally substituted with at least one substituent R¹², or (R³ and R⁴), and/or (R⁴ and R⁵), and/or (R⁵ and R⁶), and/or (R⁶ and R⁷), and/or (R⁷ and R⁸), together with the atom(s) they are attached, form a 3- to 8-membered saturated, partially or fully unsaturated ring having 0, 1 or 2 heteroatoms independently selected from —NR¹³—, —O—, —S—, —SO— or —SO₂—, and said ring is optionally substituted with at leaset one substituent R¹²,
provided that
when X is O, R⁵ and R⁶ are absent,
when X is N, R⁶ is absent, an
when X is S, R⁵ and R⁶ are absent, or at least one of R⁵ and R⁶ is oxo,
when one of R³ and R⁴ is oxo, the other is absent,
when one of R⁷ and R⁸ is oxo, the other is absent, and
when X is C and one of R⁵ and R⁶ is oxo, the other is absent;
R⁹, R¹⁰, and R¹¹, which may be the same or different, are each selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R¹² is selected from CN, halogen, haloalkyl, NO₂, —NR′R″, —OR′, oxo, —COR′, —CO₂R′, —CONR′R″, —NR′CONR″R′″, —NR′CO₂R″, —NR′SO₂R″, —SO₂R′, alkyl, alkenyl, alkynyl, cycloalkyl, heterocyclyl, aryl, and heteroaryl, wherein R′, R″, and R′″ are independently selected from hydrogen, haloalkyl, alkyl, arylalkyl, alkenyl, alkynyl, cycloalkyl, heterocyclyl, aryl, and heteroaryl, or (R′ and R″), and/or (R″ and R′″) together with the atoms to which they are attached, form a 3- to 8-membered saturated, partially or fully unsaturated ring having 0, 1 or 2 additional heteroatoms independently selected from —NR¹³—, —O—, —S—, —SO— and —SO₂—; and
R¹³ is selected from hydrogen, alkyl, cycloalkyl, aryl, heteroaryl, and heterocyclyl.

2. The method of claim 1, wherein the cancer is solid cancer.

3. The method of claim 1, wherein the cancer is selected from colorectal cancer, gastric cancer, small cell lung cancer, breast cancer, ovarian cancer, fallopian tube carcinoma, peritoneal carcinoma, melanoma, glioblastoma or lymphoma.

4. The method of claim 1, wherein the cancer is glioblastoma with unmethylated MGMT promoter.

5. The method of claim 1, wherein the cancer is recurrent/refractory glioblastoma.

6. The method of claim 1, wherein the PARP inhibitor is the compound of Formula (III), 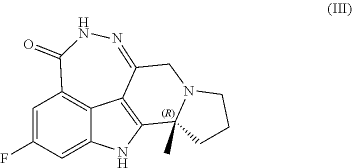
or a pharmaceutically acceptable salt thereof.

7. The method of claim 1, wherein the PARP inhibitor is the compound of Formula (IV),

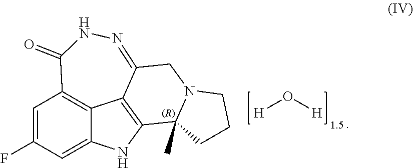

8. The method of claim 1, wherein the PARP inhibitor is administrated at a dose of 1-120 mg twice daily.

9-10. (canceled)

11. The method of claim 8, wherein temozolomide is administered orally at a dose of 20-120 mg/m²/day.

12. The method of claim 8, wherein the radiation therapy administered QD×5 days/week for 6 to 7 weeks with 1.8 to 2 Gy/fraction for a total dose of up to 60 Gy.

13. The method of claim 1, wherein the cancer is locally advanced or metastatic solid tumors or newly diagnosed or recurrent/refractory glioblastoma.

14. A pharmaceutical combination for use in the prevention, delay of progression or treatment of cancer, comprising a PARP inhibitor and temozolomide, wherein the PARP inhibitor is a compound of Formula (I), 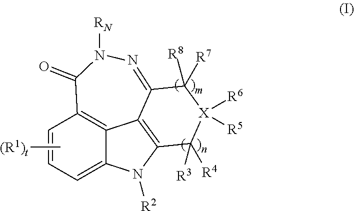
a stereoisomer thereof, a pharmaceutically acceptable salt[[s]] thereof, or a solvate thereof, wherein:
R_{N} is selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
X is selected from the group consisting of C, N, O, and S;
m and n, which may be the same or different, are each an integer of 0, 1, 2, or 3;
t is an integer of 0, 1, 2, or 3;
R¹, at each occurrence, is independently selected from halogen, CN, NO₂, OR⁹, NR⁹R¹⁰, NR⁹COR¹⁰, NR⁹SO₂R¹⁰, CONR⁹R¹⁰, COOR⁹, SO₂R⁹, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R² is selected from hydrogen, COR⁹, CONR⁹R¹⁰, CO₂R⁹, SO₂R⁹, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R³, R⁴, R⁵, R⁶, R⁷ and R⁸, which may be the same or different, are each independently selected from hydrogen, halogen, —NR⁹R¹⁰, —OR⁹, oxo, —COR⁹, —CO₂R⁹, —CONR⁹R¹⁰, —NR⁹CONR¹⁰R¹¹, —NR⁹CO₂R¹⁰, —NR⁹SO₂R¹⁰, —SO₂R⁹, alkyl, alkenyl, cycloalkyl, aryl, heterocyclyl, alkynyl, and heteroaryl, wherein each of the alkyl, alkenyl, alkynyl, cycloalkyl, aryl, heterocyclyl, and heteroaryl is independently optionally substituted with at least one substituent R¹², or (R³ and R⁴), and/or (R⁴ and R⁵), and/or (R⁵ and R⁶), and/or (R⁶ and R⁷), and/or (R⁷ and R⁸), together with the atom(s) they are attached, form a 3- to 8-membered saturated, partially or fully unsaturated ring having 0, 1 or 2 heteroatoms independently selected from —NR¹³—, —O—, —S—, —SO— or —SO₂—, and said ring is optionally substituted with at leaset one substituent R¹²,
provided that
when X is O, R⁵ and R⁶ are absent,
when X is N, R⁶ is absent, an
when X is S, R⁵ and R⁶ are absent, or at least one of R⁵ and R⁶ is oxo,
when one of R³ and R⁴ is oxo, the other is absent,
when one of R⁷ and R⁸ is oxo, the other is absent, and
when X is C and one of R⁵ and R⁶ is oxo, the other is absent;
R⁹, R¹⁰, and R¹¹, which may be the same or different, are each selected from hydrogen, alkyl, alkenyl, alkynyl, cycloalkyl, heterocyclyl, aryl, and heteroaryl, wherein each of the alkyl, alkenyl, alkynyl, cycloalkyl, heterocyclyl, aryl, and heteroaryl is independently optionally substituted with at least one substituent R¹²;
R¹² is selected from CN, halogen, haloalkyl, NO₂, —NR′R″, —OR′, oxo, —COR′, —CO₂R′, —CONR′R″, —NR′CONR″R′″, —NR′CO₂R″, —NR′SO₂R″, —SO₂R′, alkyl, alkenyl, alkynyl, cycloalkyl, heterocyclyl, aryl, and heteroaryl, wherein R′, R″, and R′″ are independently selected from hydrogen, haloalkyl, alkyl, arylalkyl, alkenyl, alkynyl, cycloalkyl, heterocyclyl, aryl, and heteroaryl, or (R′ and R″), and/or (R″ and R′″) together with the atoms to which they are attached, form a 3- to 8-membered saturated, partially or fully unsaturated ring having 0, 1 or 2 additional heteroatoms independently selected from —NR¹³—, —O—, —S—, —SO— and —SO₂—; and
R¹³ is selected from hydrogen, alkyl, cycloalkyl, aryl, heteroaryl, and heterocyclyl.

15. The pharmaceutical combination of claim 14, wherein the cancer is solid cancer.

16. The pharmaceutical combination of claim 14, wherein the cancer is selected from colorectal cancer, gastric cancer, small cell lung cancer, breast cancer, ovarian cancer, fallopian tube carcinoma, peritoneal carcinoma, melanoma, glioblastoma or lymphoma.

17. The pharmaceutical combination of claim 14, wherein the cancer is glioblastoma with unmethylated MGMT promoter.

18. The pharmaceutical combination of claim 14, wherein the cancer is recurrent/refractory glioblastoma.

19. The pharmaceutical combination of claim 14, wherein the PARP inhibitor is the compound of Formula (III), 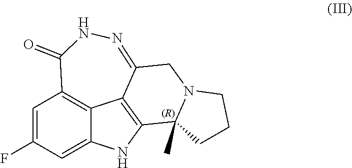
or a pharmaceutically acceptable salt thereof.

20. The pharmaceutical combination of claim 14, wherein the PARP inhibitor is the compound of Formula (IV),

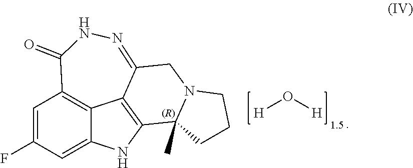

21. The pharmaceutical combination of claim 14, wherein the PARP inhibitor is administrated at a dose of 1-120 mg twice daily.

22-23. (canceled)

24. The pharmaceutical combination of claim 21, wherein temozolomide is administered at a dose of 20-120 mg/m²/day.

25. The method of claim 11, wherein temozolomide is administered orally at a dose of 23, 46 or 69 mg/m²/day.

26. The pharmaceutical combination of claim 24, wherein temozolomide is administered at a dose of 23, 46 or 69 mg/m²/day.
